(19) United States
(12) Patent Application Publication
(10) Pub. No.: US 2025/0050067 A1
(43) Pub. Date: Feb. 13, 2025

(54) HYDRAULICALLY ACTUATED CATHETER LIKE SYSTEM FOR TREATING VASCULAR AND NON-VASCULAR DISEASES

(71) Applicant: BIOTRONIK AG, BUELACH (CH)

(72) Inventors: Azadeh Mehrabi, Meilen (CH); Jeremy Wernli, Wettingen (CH); Matthias Wesselmann, Ruedlingen (CH); Bodo Quint, Dettighofen (DE)

(21) Appl. No.: 18/721,814

(22) PCT Filed: Jan. 16, 2023
(86) PCT No.: PCT/EP2023/050840

§ 371 Date: Jun. 19, 2024

(30) Foreign Application Priority Data
Jan. 17, 2022 (EP) 22151791.5

(51) Int. Cl. A61M 25/01; A61F 2/95; A61M 25/00; A61M 25/09

(52) U.S. Cl. CPC A61M 25/0122; A61F 2/95; A61M 25/0082; A61M 25/09; A61M 2025/09133

(57) ABSTRACT

A catheter includes a rolling membrane defining an inner volume that can be pressurized. The rolling membrane is configured to roll out in a longitudinal direction (D) into a rolled-out state. An inner shaft is attached to the rolling membrane. The membrane can roll out in a longitudinal direction along a vessel of a patient.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/050840, which was filed Jan. 16, 2023, which application claimed priority from EP Application 22151791.5, which was filed Jan. 17, 2022.

### FIELD OF THE INVENTION

A field of the invention concerns devices and systems for treating vascular and non-vascular diseases.

### BACKGROUND

Over the last years, the annual number of necessary vascular interventions has increased. This may at least in part be explained by an aging population and disadvantageously changing lifestyle habits (e.g., fat-rich nutrition) of people which may both contribute to an increase in vascular diseases/lesions which may then oftentimes require the aforementioned vascular interventions. Not only the annual number of necessary vascular interventions has increased but also the range of indications (e.g., the number of different vascular diseases which require an intervention) and the complexity of required vascular interventions has increased, thus increasing the burden on the associated medical staff. Besides routine vascular interventions, as a prominent, but non-exhaustive, representative of vascular interventions, percutaneous coronary interventions (PCIs) may be named. PCIs may be based on percutaneously inserting catheters into the vascular system of the patient and propagating the catheters forward, through the vascular system of the patient, to the location of the vascular disease. However, during said conventional PCIs, physicians often encounter, as part of the increased complexity of vascular interventions, uncrossable lesions which cannot be crossed with a guidewire (as the most common cause of technical failure) and/or which cannot be crossed with a balloon catheter (as the second most common cause of technical failure). The aforementioned complexities are commonly encountered in tortuous and calcified arteries as well as in chronical total occlusions (CTOs). Even in scenarios, in which a crossing of the lesion with the guidewire may be possible (in routine and/or PCI interventions), it may still be possible that the lesion itself cannot be crossed with the balloon catheter. This may increase complexity of the PCIs and the associated time efforts for the medical staff. Further drawbacks arising from the aforementioned technical failures may be seen in an increase of the radiation dose a patient may be exposed to (e.g., to localize a current position of the catheters), exposure to increased contrast volumes accompanied by a general lower likelihood of procedural success.

As a further drawback, in conventional angioplasty of complex coronary artery lesions, the coronary intervention is typically performed in a two-step process including the insertion of a guidewire and/or a guiding catheter followed by the insertion of a second balloon catheter and/or a stent delivery system which may be pushed along the guidewire. Therefore, at least two catheters are required which are inserted into the patient's body in at least a two-step process thus increasing the required number of medical instruments necessary for the vascular intervention as well as an increase in procedural time required to perform the vascular intervention. Moreover, the selection of the guidewire is typically seen as a paramount requirement for successful angioplasty with stringent requirements on the guidewire, which must be sufficiently flexible and stiff enough for kink resistance and pushability through the artery system of the patient. This additional task of adequately selecting the guidewire is carried out by a physician in charge of a conventional vascular intervention with respective effects on the overall procedural time required to perform the vascular intervention. In percutaneous transluminal coronary angioplasty (PTCA) a guiding catheter is usually inserted into the access sheath together with the guidewire, once the guiding catheter is in place, the guidewire is pushed out of the guiding catheter and inside the coronaries and through the stenosis and then the guide extension catheter is introduced into the guiding catheter and find its way into the vessel by following the guidewire. A typical guide extension catheter may be a monorail system. In a guide extension catheter (GEC) or guide catheter extension a guide extension (e.g. having a length of 25 cm) may be passed over the guiding catheter in a rapid exchange fashion and may extend beyond the distal end of the guiding catheter. The guide extension catheter may be thinner than the guiding catheter by, e.g., 1 Fr and it may be designed to minimize trauma to the coronary artery. The proximal end of the guide extension catheter may be attached to a thin stainless-steel pushrod, which may be used to push and pull the guide extension catheter independent of the guiding catheter. However, even modern guide extension catheters still have difficulties crossing calcified arteries, in particular, when 180° bends are present, due to excessive friction forces. Physicians may then use a balloon catheter to help the guide extension catheter to cross such a bend. This procedure requires placing the balloon distally from the guide extension catheter followed by an inflation of the balloon and a subsequent pushing of the guiding catheter over the balloon catheter while deflating the balloon catheter. However, in some cases, the balloon catheter and the guiding catheter may move relative to the vessel wall and may cause injuries to the intima due to excessive shear.

In view of the plurality of drawbacks physicians commonly encounter during conventional vascular interventions known in the art, there is a need for improving conventional vascular interventions to overcome at least some of these drawbacks at least in part.

### SUMMARY OF THE INVENTION

AA preferred catheter includes a rolling membrane (hereinafter also called rolling membrane catheter). The catheter is configured to cross a stenosis or a chronic total occlusion. The rolling membrane defines an inner volume that can be pressurized, the rolling membrane being configured to roll out in a longitudinal direction (D) into a rolled out state. The catheter includes an inner shaft and/or an outer shaft, and a catheter length of 80 cm to 200 cm. The rolling membrane has an outer diameter of between 1.5 mm to 5.0 mm and the rolling membrane has a length of between 4 cm to 60 cm.

A preferred embodiment is a catheter that includes a rolling membrane defining an inner volume that can be pressurized. The rolling membrane is configured to roll out in a longitudinal direction (D) into a rolled-out state. An inner shaft is attached to the rolling membrane. A medical device and/or medical device delivery system and/or a drug and/or a drug delivery system and/or a contrast agent and/or an aspiration system is arranged within the inner shaft.

### BRIEF DESCRIPTION OF THE DRAWINGS

The following figures are provided to support the understanding of the present invention:

FIGS. 1a-c Illustration of a rolling membrane catheter without inner/outer shaft and with a same diameter as a vessel of a patient;

FIG. 2a-c Illustration of a rolling membrane catheter without inner/outer shaft and with a smaller diameter than a vessel of a patient;

FIG. 3a-c Illustration of a rolling membrane catheter with inner shaft and without outer shaft and with a same diameter than a vessel of a patient;

FIG. 4a-c Illustration of a rolling membrane catheter with inner shaft and without outer shaft and with a smaller diameter than a vessel of a patient;

FIG. 5a-c Illustration of a rolling membrane catheter with outer shaft and without inner shaft and with a same diameter than a vessel of a patient;

FIG. 6a-c Illustration of a rolling membrane catheter with outer shaft and without inner shaft and with a smaller diameter than a vessel of a patient;

FIG. 7a-c Illustration of a rolling membrane catheter with outer shaft and with inner shaft and with a same diameter than a vessel of a patient;

FIG. 8a-c Illustration of a rolling membrane catheter with outer shaft and with inner shaft and with a smaller diameter than a vessel of a patient;

FIG. 9a-c Illustration of exemplary rolling membrane surface designs;

FIG. 10 Illustration of an exemplary helix-like rolling membrane design;

FIG. 11a-c Illustration of an exemplary tapered membrane design;

FIG. 12a-d Illustration of an exemplary stepped membrane design;

FIG. 13a-d Illustration of a rolling membrane-based balloon angioplasty;

FIG. 14a-b Illustration of a rolling membrane-based drug coated balloon angioplasty;

FIG. 15a-b Illustration of a rolling membrane-based CTO crossing;

FIG. 16a-b Illustration of a rolling membrane-based local drug release/object aspiration;

FIG. 17a-e Illustration of a rolling membrane-based flexible balloon catheter delivery system;

FIG. 18a-d Illustration of a rolling membrane-based preloaded balloon catheter delivery system;

FIG. 19 Illustration of a rolling membrane-based microcatheter delivery system;

FIG. 20a-e Illustration of a rolling membrane-based SX-stent delivery system;

FIG. 21a-e Illustration of a rolling membrane-based flexible BE-stent delivery system;

FIG. 22a-d Illustration of a rolling membrane-based preloaded BE-stent delivery system;

FIG. 23a-b Illustration of a rolling membrane-based dual-lumen catheter for atherectomy;

FIG. 24a-e Illustration of a special guidewire steering concept for rolling membrane catheters;

FIG. 25a-g Overview of potential locations of a lesion.

### DETAILED DESCRIPTION OF THE PREFERRED EMBODIMENTS

The rolling membrane may be adapted to roll in a longitudinal direction along a vessel of a patient, for example by at least five centimeters. By a rolling membrane, which may extend e.g. by at least five centimeters, the steering of the catheter may be simplified, as the rolling membrane may to a certain extent be capable of unfolding itself along the vessel without any guidance requirements supplied by a physician and/or the presence of a guiding catheter which may have been inserted into a vessel of the patient in a preceding step of a vascular intervention. Therefore, said aspect of the present invention may allow for an “all-in-one”-catheter solution. This may in particular simplify the overall vascular intervention by decreasing its complexity as the steering requirements, to be supplied by a physician, may be reduced and the procedural steps may be minimized (e.g., as no guiding catheter may be required). Further, the use of a (long) rolling membrane may provide the catheter with increased flexibility over a (long) longitudinal range while at the same time ensuring good deployability.

A rolling membrane may be understood as forming at least a portion of a (flexible) hose that may be arranged essentially along a longitudinal direction of the vessel in a rolled out state. The rolling membrane or hose preferably possesses a circular shaped cross section (as seen from a cut perpendicular to a longitudinal extension of the hose), a first end and an opposing second end (with respect to a longitudinal/length extension of the hose). The second end may be attached to an external shaft or it may extend to a position outside the patient. The first end may be sealed. In a rolled out state, the rolling membrane or hose may thus form an inner volume, extending from the first end towards a proximal direction that may be encompassed by a circumferential, e.g. cylindrical, (flexible) wall of the hose. At least a portion of the rolling membrane or hose (e.g. at the first end) may be foldable inwards, such that at least a portion of the hose (e.g. of its outer wall) may be retracted into the catheter. By retracting and/or pushing forward (of e.g. the second end) and/or pressurizing the inner volume formed by the rolling membrane the rolling membrane may be retracted (proximally) and/or moved forward (distally) in an essentially friction-less manner (concerning the vessel). A rolling of the rolling membrane may thus be understood as a corresponding folding outwards (or inwards) of at least a portion of the hose, e.g., in a longitudinal direction along a vessel of the patient. It may be possible that the rolling membrane is adapted to roll out by more than 10 cm, more than 20 cm, up to 30 cm or even up to 40 cm or more in a longitudinal direction, e.g. along an ostium (as measured from an ostium), during an investigation and/or medical treatment such as, e.g., a vascular intervention. Hence, an essentially frictionless deployment of the catheter may be provided, optionally even without an outer shaft (the long rolling membrane may replace the shaft) and/or without an inner shaft (a simple wire may be used for moving the second end of the rolling membrane) and/or without a guidewire.

The rolling membrane catheter may include an inner shaft and/or an outer shaft. However, a rolling membrane catheter with or without an inner shaft and/or an outer shaft may be provided.

The inner shaft and/or the outer shaft may be reinforced preferably by a metal, metal alloy or polymer braid. Thus, the inner shaft and/or the outer shaft may be a braided inner and/or outer shaft or a fiber reinforced inner and/or outer shaft.

The concept of a rolling membrane may not be limited to vascular interventions only but may also be used in neurological catheters (neurovascular field), in oncology, in ear, nose, and throat medicine, in endoscopy, in urology, gastroenterology, gynecology, pulmonology or in the nasolacrimal duct.

Another aspect of the present invention relates to a catheter which may additionally or alternatively include: a rolling membrane defining an inner volume that can be pressurized and/or a guiding element arranged within the inner volume for guiding the rolling membrane along a vessel of a patient. The guiding element could be any element which enables a pushing of the rolling membrane. The guiding element may be a guidewire, e.g. made of a (suitable) metal or metal alloy or a (suitable) polymer.

It may be possible that the second (distal) end of the rolling membrane (e.g. of the hose forming the rolling membrane) may temporarily or permanently be sealed such that an inner volume of the hose is formed. It may thus be facilitated that a fluid, e.g. a (pressurized) gas and/or a (pressurized) liquid, can be inserted into the inner volume of the rolling membrane, e.g., via the first end of the membrane (or the hose forming the membrane).

The inner volume of the rolling membrane may additionally or alternatively be provided with a guiding element to allow additional steerability. The additional steerability may in particular be beneficial in situations in which the rolling membrane has to pass one or more vascular branches prior to its arrival at a lesion to be processed. The guiding element may exit the rolling membrane through the first (proximal) end of the hose associated with the rolling membrane and may be adapted to be manipulated by a physician from outside of the body of the patient.

Therefore, by the inventive catheter, an additional guiding catheter may be avoided thus simplifying the vascular intervention and decreasing the number of medical instruments required.

The guiding element may be adapted to be slidable with respect to an inner side of the rolling membrane, such that the guiding element can remain at a distal portion of the rolling membrane as the rolling membrane is rolled out. By adapting the guiding element such that it is slidable with respect to an inner side of the rolling membrane, it may be facilitated that the region in which the guiding element may be in contact with the rolling membrane remains at a location of the rolling membrane at which the steerability of the rolling membrane by the guiding element is maximized, e.g., the distal portion of the rolling membrane, regardless of the exact amount to which the membrane has been rolled out. The sliding back of the guiding element could be enabled by applying a pulsating hydraulic pressure. During non-pressurized or low pressurized phase of the rolling membrane a guiding element under tension (e.g. anchored with a spring like element outside the patient) can automatically pull the guiding element back.

A distal portion of the rolling membrane may be understood as a portion of the rolling membrane which is further apart from a physician manipulating the catheter as an opposing, with respect to a longitudinal extension of the catheter, proximal portion of the rolling membrane which may be closer to the physician manipulating the catheter. In a fully rolled out state, the distal portion will include the second end of the membrane. In only partially rolled out states, the second end of the membrane will to some extent be retracted and thus generally be located proximally with respect to the distal portion of the membrane.

In some scenarios in which the rolling membrane is adapted to roll out only by a short distance (e.g., less than 5 cm), it may alternatively be possible to adapt the guiding element such that it is jointly connected to the inner side of the rolling membrane (instead of slidably arranged).

The guiding element may include a curved wire. Providing the guiding element with a curved wire may facilitate easier steerability of the rolling membrane as a physician, manipulating the curved wire from outside the patient's body, may transfer a certain force onto the distal portion of the rolling membrane by, e.g., rotating the curved wire by a certain angular amount about the longitudinal axis of the curved wire.

The transfer of the force may be optimized for the respective needs of the vascular intervention by selecting the curvature of the wire accordingly, e.g., a larger curvature may be associated with a larger force transfer onto a distal portion of the rolling membrane as compared to a wire with less curvature. In some scenarios, the curvature may be non-uniform along a longitudinal extension of the curved wire, i.e., the effective bending radius may vary in between adjacent infinitesimal small portions of the curved wire. The guiding element may include the curved wire and may additionally include a sliding element which slidable contacts the rolling membrane such that a force transfer of the curved wire and the guiding element is facilitated.

Another aspect of the present invention relates to a catheter which may additionally or alternatively include: a rolling membrane which may include a retraction element for retracting the rolling membrane from a rolled out state of the rolling membrane. The retraction element may not include an inner lumen. The retraction element may always be under tension and does not need to be made of a pushable material. The retraction element can be any element with a sufficient tensile strength to stop the eversion of the rolling membrane and the retraction element must allow to pull the rolling membrane back into the shaft. Therefore, the retraction wire could be a yarn, a filament or a (fine) rod.

By providing the rolling membrane of the catheter with a retraction element, it may be facilitated that the rolling membrane may easily be retracted (from outside the patient's body) if necessary. A potential depressurizing of the rolling membrane alone may not in all scenarios be sufficient to easily and/or fully retract the rolling membrane. By abstaining from providing the retraction element with an inner lumen, the retraction element may be implemented as a piece of solid wire which may increase the flexibility of the retraction element and may reduce its weight.

In one embodiment an end portion of the membrane may be adapted to form a distal tip of the catheter when the rolling membrane is rolled out. The end portion may include the first end of the membrane. Since said end portion may be folded inwards into the rolling membrane, in an inwards folded state (i.e., a state in which the rolling membrane is not rolled out), the overall dimensioning of the catheter, and in particular of the rolling membrane, may be minimized. Therefore, and undue handling difficulties which may potentially arise from a long rolling membrane may thus be avoided by the compact design.

The retraction element may be attached to the end portion of the rolling membrane. By attaching the retraction element to the end portion of the rolling membrane an optimized retraction of a rolled out portion of the rolling membrane may be facilitated. Preferably, the end portion of the rolling membrane (which may be associated with the first end of the hose as outlined above) may be the portion of the rolling membrane which is rolled out lastly. Therefore, the end portion is the portion of the rolling membrane which may be retracted firstly. This may avoid any undesired overlap of the side walls of the rolling membrane as they are folded inwards/retracted. This contributes to a simplified handling of the catheter.

The retraction element may be adapted to steer the rolling membrane along a vessel as it is rolled out. By adapting the retraction element such that it may steer the rolling membrane, the complexity of the catheter may further be reduced as it may also be possible to (exclusively) steer the rolling membrane (solely) by the retraction element as it is rolling out along in a longitudinal direction along a vessel of a patient (e.g. without the need for a guidewire).

In some scenarios, the retraction element may include a curved wire or at least a wire with curved distal end. In such a case, the direction along which an expansion of the rolling membrane occurs may be determined by a physician from outside the patient's body.

In some scenarios the retraction element may be adapted to additionally support the guiding of the rolling membrane besides an (optional) guiding element. If the retraction element and the guiding element are in contact with the rolling membrane at two different locations, additional degrees of freedom may be provided for steering the rolling membrane. Therefore, the flexibility and steerability of the catheter may be improved.

In some scenarios, an outer diameter of the catheter (and/or the rolling membrane) may be smaller than an inner diameter of the vessel of the patient.

By providing the catheter with a diameter which may be smaller than the inner diameter of the vessel of the patient, it may be ensured that blood may easily pass the catheter as the catheter propagates to, e.g., a lesion to be processed. By ensuring sufficient blood flow passing the catheter, a tissue or patient trauma further distal of the most distal portion of the catheter may be avoided which may in particular be relevant if time intensive vascular interventions are necessary. Moreover, by providing the catheter with a diameter smaller than the diameter of the vessel of the patient, easier propagation of the catheter along the vessel of the patient may be facilitated.

The catheter may be adapted to acquire a helix-like or helical shape along a longitudinal direction of the vessel of the patient. For example, a radius of the helix and/or a pitch of the helix can be non-uniform.

If the catheter is adapted to acquire a helix shape along a longitudinal direction of the vessel of the patient, it may be facilitated that the rolling membrane of the catheter may be in contact with the inner walls of the vessel of the patient at one or more distinct locations (e.g., to ensure an at least local immobilization of the catheter) while still allowing sufficient blood passing by the catheter. As outlined above, this may avoid a tissue or patient trauma further distal from the catheter.

A helix may be understood to include a corkscrew shape and shapes similar to a spiral staircase. The radius and/or pitch of the helix may vary smoothly along a longitudinal axis of the vessel. The radius of the helix may be understood as an inner radius, e.g., as the shortest distance from the longitudinal axis of the vessel, about which the helix is twined, to the longitudinal center axis of the catheter, i.e., the longitudinal center axis of the rolling membrane, forming the helix about the longitudinal axis. The pitch of the helix may be understood as the height of one complete helix turn, measured parallel to the longitudinal axis of the helix.

In an alternative scenario it may be possible that at least one of the radii of the helix and/or the pitch of the helix are uniform (i.e., constant) along a longitudinal extension of the catheter.

In some scenarios it may be possible that the rolling membrane intrinsically possesses a tendency, e.g., due to a specific manufacturing process, to form the above-mentioned helix-like shape and/or that the helix-like shape can be acquired (or strengthened) as a result of a physician manipulating (e.g. twisting, rotating and/or retracting the catheter), preferably from outside the patient's body. For example, if the longitudinal extension of the rolling membrane is hindered, the rolling membrane is forced to take a helical shape. The stronger the tension on the retraction element during eversion the more helical would be the shape of the rolling membrane. The helical shape of the rolling membrane could be also obtained by thermoforming the rolling membrane in a helical shape. The helical shape of the rolling membrane could be also obtained by intentional inhomogeneous circumferential wall-thickness distribution, e.g. which was induced during an extrusion process of the rolling membrane. Additionally, the tubing may be rotated while pulling the tubing. From which the rolling the rolling membrane will be thermoformed out of the die to achieve a helical shape.

Another aspect of the invention relates to a catheter, wherein the catheter may include a rolling membrane which may define an inner volume that can be pressurized. The rolling membrane may additionally or alternatively to the further aspects described herein be adapted such that, by pressurizing the inner volume, a stenosis in a blood vessel of a patient may be dilated via dilation by the rolling membrane.

By providing the catheter with a rolling membrane which may define an inner volume which can be pressurized, it may be facilitated that the rolling membrane may apply a radial force onto an inner wall of the blood vessel of the patient at a location at which the rolling membrane is in contact with the blood vessel. By sufficiently pressurizing the inner volume of the rolling membrane, an at least local dilation of the blood vessel may be facilitated which may allow an at least local removal of a stenosis at the dilated location of the blood vessel of the patient.

In general the rolling membrane is configured such that the rolling membrane to a larger extent extends in the longitudinal direction when being pressurized, and only to a smaller extent along a radial direction (e.g., perpendicular to a longitudinal direction along which the rolling membrane is rolled out). In some cases, it may be possible that the rolling membrane is configured such that it may (essentially) only extend in the longitudinal direction when it is rolled out by a certain amount of its maximum length (e.g., if it is rolled out by at least 20%, 30%, 40%, 50%, 60%, 70%, 80%, 90% of its total length).

However, in one embodiment it may be possible that the rolling membrane is adapted in addition such that (at least) a portion of a rolled out portion of the rolling membrane predominantly (i.e., to a larger extent) expands in a radial direction (e.g., perpendicular to a longitudinal direction along which the rolling membrane is rolled out), and only to a smaller extent along a longitudinal direction. In some cases, it may be possible that the rolling membrane is adapted such that (at least) a portion may essentially only expand radially when it is rolled out by a certain amount of its maximum length (e.g., if it is rolled out by at least 20%, 30%, 40%, 50%, 60%, 70%, 80%, 90% of its total length).

An outer diameter of the catheter (e.g. the rolling membrane) may be adapted to essentially fill the vessel of the patient in a radial direction. By providing the catheter, such that it may essentially fill the vessel of the patient, an immobilization of the catheter relative to the vessel at a certain location may be facilitated. This may, e.g., allow a defined pressure transfer from the rolling membrane in a radial direction onto a certain location of an inner wall of the vessel such as to at least locally dilate a stenosis. Moreover, the local immobilization may ensure that subsequent treatment steps may be prepared and/or executed at the location of immobilization.

An essential filling of the vessel of the patient may be understood as a scenario in which the catheter has approximately (neglecting minor deviations arising from non-uniform vessel diameters relative to non-uniform catheter diameters as they arise from manufacturing tolerances of the catheter) the same diameter as the vessel of the patient (at least at a certain location).

It may be possible that the catheter itself essentially fills the vessel of the patient (i.e., in a state in which the rolling membrane is not yet (fully) rolled out) and/or that the catheter essentially fills the vessel of the patient as a consequence of rolling out the rolling membrane by a certain amount and a subsequent radial dilation.

The rolling membrane may be adapted such that a proximal end of the rolling membrane can be located outside of a body of a patient, as the rolling membrane is rolled out inside the body of the patient. By providing the rolling membrane such that it may extend to the outside of the body of the patient, the complexity of the catheter may further be reduced as the rolling membrane may be the only part of the catheter which extends along the vascular system of the patient.

In such a scenario, the catheter may optionally not be provided with any additional catheter elements which may deliver the rolling membrane to a certain location in the vessel of the patient at which the rolling out of the rolling membrane may be initiated.

The rolling membrane may at least partially be provided with a non-uniform outer surface. For example, the non-uniform outer surface may be adapted to at least locally increase friction to the vessel of the patient and/or to at least locally resect a stenosis. By providing the rolling membrane with a non-uniform outer surface, the frictional properties of the rolling membrane, when being in contact with the vessel of the patient, may be locally increased or decreased which may provide tailored immobilization capabilities (e.g., a designated area on the rolling membrane may be provided with a surface which may provide higher or lower friction as compared to an adjacent designated area) of the rolling membrane within a vessel of the patient. Moreover, the non-uniform outer surface may also be used as a medical tool to at least locally remove a stenosis (similar to the concept of a scoring balloon or cutting balloon) arising from a contact of the non-uniform outer surface of the rolling membrane and the stenosis if the rolling membrane is pressurized. By applying a pressure to the rolling membrane peaks or spikes being arranged on the outer surface of the rolling membrane may break a stenosis due to local pressure maxima. The non-uniform outer surface may also be used for sticking the rolling membrane to the vessel wall, thereby giving the rolling membrane support for advancing further into the vessel. The non-uniform outer surface may include a pattern such as protrusions and/or recesses.

The pattern may cover the entire outer surface of the rolling membrane. As an alternative, the non-uniform outer surface may include one or more than one distinct surface region which may be provided with respective different patterns. Additionally or alternatively, only a fraction of the total surface of the rolling membrane may be provided with a pattern.

Additionally or alternatively, a radius and/or a diameter of the rolling membrane, in a rolled out state, may vary along a longitudinal direction of the rolling membrane. By providing the rolling membrane with a radius and/or a diameter which may be non-uniform along a longitudinal direction of the rolling membrane, it may be facilitated to access portions of the vessel of the patient which taper. In such a case, a distal portion and/or a distal end of the rolling membrane may be provided with a smaller radius as compared to a portion of the rolling membrane which may be located at a further proximal location. Thus, the distal portion or distal end may be capable of entering a narrower region of the vessel as compared to the further proximal portion of the rolling membrane. The portion of the rolling membrane which is provided with the larger radius may be adapted such that it may be used to locally immobilize the rolling membrane in front of a tapering region of the vessel. A rolling membrane with a non-uniform diameter along its longitudinal direction may reach an arbitrary diameter at a certain position in the vessel if it is not only rolled in and out but also axially displaced in addition. Thus, once the rolling membrane diameter gets too large for the vessel in a typical modus operandi a roll out of the rolling membrane is stopped, the rolling membrane is (at least partially) deflated, the catheter is pulled back and the rolling membrane is roll out again.

In some scenarios it may also be possible that the distal-most portion of the rolling membrane is provided with a larger diameter as compared to a region of the rolling membrane which is located further proximal.

Another aspect relates to a catheter which may additionally or alternatively include a rolling membrane adapted to be rolled out such as to form an elongated main portion and a distal end portion. The elongated main portion may be adapted to have a non-uniform cross-section. By providing the elongated main portion with a non-uniform cross section, the rolling membrane may enter tapering regions of the vessel of the patient. In some scenarios, the rolling membrane may be adapted such that the diameter associated with the non-uniform cross section may be varied by changing the extent to which the rolling membrane is rolled out. In some scenarios, the diameter of the rolling membrane may increase the further the rolling membrane is rolled out. In other scenarios, the diameter of the rolling membrane may decrease the further the rolling membrane is rolled out.

Another aspect relates to a catheter wherein the catheter may include a rolling membrane, wherein, additionally or alternatively, at least a portion of the rolling membrane can be folded inwards forming an inner side wall and wherein the inner side wall may include a drug layer. The drug layer may be adapted such that it is transported distally by rolling out the rolling membrane (and may then form part of an outer surface of the rolling membrane). By providing the rolling membrane with a drug layer it may be facilitated to deliver a drug inside the rolling membrane to a certain location in the human or animal body, e.g. in the vessel of the patient (protected by the rolling membrane). By folding the drug layer outwards, the drug may be released at the desired location in the human or animal body, e.g. in the vessel of the patient. Therefore, targeted drug delivery in the human or animal body, e.g. inside a vessel of the patient, may be facilitated and the medical effect of the respective drug may only be provided at said desired location. Furthermore, the length of the area on which the drug is applied in the human or animal body, e.g. to a vessel, can be varied by rolling out the rolling membrane more or less. Moreover, a dilution of the drug (therefore suppressing its medical effect), as a result of a mixture of the drug with blood, may significantly be reduced.

Another aspect of the present invention relates to a catheter, wherein the catheter may include a rolling membrane, wherein the rolling membrane may additionally or alternatively be provided with a non-uniform outer surface.

An aspect relates to a catheter which may include a rolling membrane which may be adapted to be rolled out such as to form an elongated main portion having a cross-section which may be smaller than a cross-section of a vessel. The rolling membrane may further be adapted such that a center line of the elongate main portion is adapted to twine around a center line of the vessel. This may allow that the elongated main portion essentially requires a helix-shaped curvature about the center line of the vessel, thereby allowing for an improved anchoring of at least the elongated main portion of the rolling membrane while still allowing sufficient blood flow in the vessel of the patient.

In some scenarios it may be possible that the rolling membrane further forms a distal portion which is not twined about a center line of the vessel but may instead extend straight along a longitudinal direction of the vessel of the patient.

Still another aspect relates to a rolling membrane catheter, wherein the catheter does not include an inner shaft. By providing a catheter without an inner shaft, the complexity of the catheter may further be reduced as no (typically rather rigid/stiff) inner shaft is needed. Therefore, the flexibility and the steerability of the catheter may be increased and the handling of the catheter may be simplified.

Another aspect relates to a catheter which may include a rolling membrane, wherein the rolling membrane may, additionally or alternatively, be adapted to apply a force onto a functional element placed in a lumen at least in part formed by an inward-folded portion of the rolling membrane. By adapting the rolling membrane to apply a force onto a functional element placed in a lumen of the rolling membrane, the functional element may be, protected by the rolling membrane, delivered to a certain location inside the vessel of the patient and released at the respective location, e.g., as a result of the force applied to the functional element. The functional element may be a piece of (guide) wire, a stent, a microcatheter, etc.

The rolling membrane may further be adapted such that the force may push the functional element in a distal direction of the catheter when the rolling membrane is at least partially rolled out in the distal direction.

Since the rolling membrane may further be adapted to a apply a force onto the functional element, obstacles (e.g., a stenosis) in the vessel of the patient may be overcome by the functional element as a result of an additionally supplied, e.g., holding or pushing force of the rolling membrane onto the functional element. Therefore, even a stenosis which could not have been penetrated by a catheter known in the prior art, may now be overcome. If the distal end of the rolling membrane runs into an obstacle, a functional element (e.g. a guidewire) may advance faster than the everting rolling membrane, thus pulling the everting rolling membrane via friction into the gap already crossed by the functional element. This enables the treatment of complex lesions. The force applied by the rolling membrane onto the functional element may be oriented along a radial (holding force) and/or a longitudinal direction (pushing force).

A rolling membrane may have a wall thickness of 10 μm to 90 μm, preferably 15 μm to 60 μm, 70-90 μm. The outer shaft may have a wall thickness of 100 μm to 500 μm.

A rolling membrane may be fabricated by blown film extrusion, by blow molding of extruded tubes or by microextrusion. It is also possible to attach an elastomeric tube to an inner and outer shaft (e.g. by gluing or welding) and pulling the elastomeric tube back into the outer shaft, thereby creating a compliant rolling membrane.

A further aspect relates to a method for dilating a stenosis in a vessel. The method may include inserting a catheter having a rolling membrane into the vessel. Moreover, the method may include the step of rolling out the rolling membrane at least in part such as to place the rolling membrane in a proximity of the stenosis. Moreover, the method may include pressurizing the rolling membrane such that the stenosis is dilated. By rolling out the rolling membrane at least in part to place the rolling membrane in a proximity of the stenosis, the rolling membrane may extend itself into the actual stenosis as a result of a further rolling out of the rolling membrane and the subsequent pressurization. Therefore, less external push force is required to guide the rolling membrane into the location of the stenosis and the actual stenosis and a predominantly radially acting force, raised as a result of pressurizing the rolling membrane, may be used to support the dilation of even a strongly pronounced stenosis.

Another aspect relates to a method for forcing a guiding element into a CTO region of a vessel. The method may include inserting a catheter having a rolling membrane into the vessel, wherein at least a portion of the guiding element is placed in a lumen at least in part formed by an inward-folded portion of the rolling membrane. A pressurizing of the rolling membrane may provide a holding force, keeping the guiding element in place (and possibly preventing it from buckling). Moreover, the method may include rolling out at least a portion of the rolling membrane in a distal direction of the catheter, such that the guiding element is forced in the distal direction and into the CTO region.

The following detailed description outlines possible exemplary embodiments of the invention.

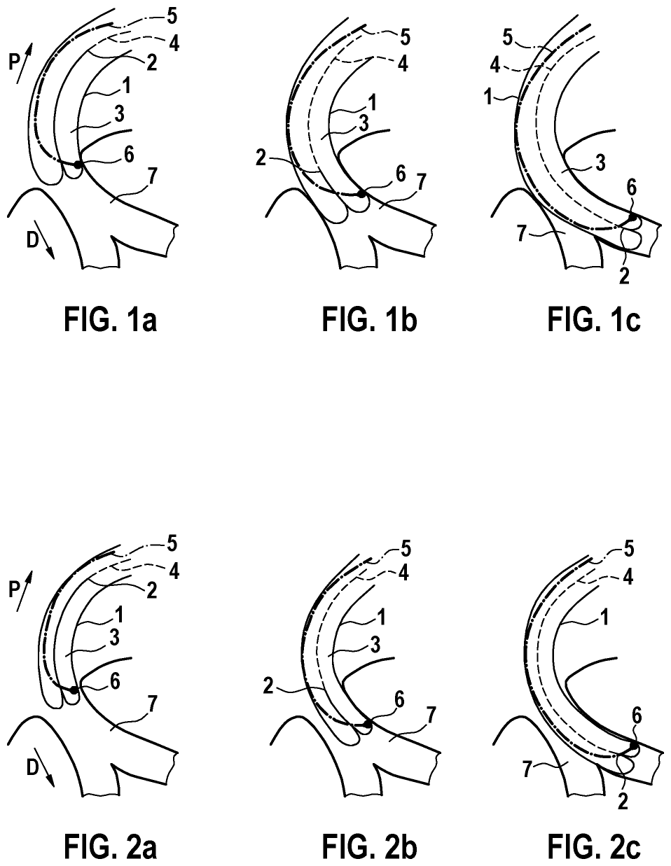

FIGS. 1a-1c show a first possible embodiment of a catheter. In this first embodiment, the catheter may be provided with a diameter with a same size as a vessel of a patient into which the catheter may be inserted.

FIG. 1a shows a catheter, located in an aorta of the patient, including a rolling membrane 1 in an initial state. In the exemplary initial state, the rolling membrane 1 may be in a state in which at least a portion of the rolling membrane may be folded inwards in itself (similar to a hose, wherein at least a portion of one end of the hose is folded inwards in itself, as further described above) by a certain amount. The reference numeral 2 refers to the point at which the retraction element 4 is joined to the first end of the rolling membrane 1. The rolling membrane 1 may be implemented such that it forms an inner volume 3. The rolling membrane 1 may be rolled out by applying a (hydraulic) pressure to the inner volume 3, e.g. by a gas or liquid. As a result of the applied pressure (which may be applied to the rolling membrane 1 from a proximal direction P), the rolling membrane 1 may roll itself out in a distal direction D. The physician may adjust the inflation (the rolling out) (and/or also the deflation, retraction) of the rolling membrane 1 according to the needs of the intervention.

The catheter may further include a retraction element 4, e.g. including a wire (e.g., without an inner lumen). At the most inwards folded location of the rolling membrane 1, the rolling membrane may be connected to the retraction element 4. The retraction element 4 may extend to the outside of the patient's body. As a result of pulling the retraction element 4 (e.g., from outside the body of the patient) in the proximal direction P, at least a portion of the rolling membrane 1, which may have been rolled out in the distal direction D, may be retracted into an at least partially inwards folded state again. Similarly, a pushing in distal direction D may lead to a further extension of the rolling membrane distally, at least when a suitable pressure is applied. Therefore, a physician may, e.g., control the length of the rolling membrane 1 which is rolled out by the retraction element 4 and/or applying pressure. As outlined above, under some circumstances, the retraction element 4 may also at least contribute to the steerability of the rolling membrane 1. Under some circumstances it may be advantageous to deflate the rolling membrane 1 at least partially. By pushing the retraction element 4 in the distal direction D, the rolling membrane 1 may be steered in a desired direction.

The catheter may further include a guiding element 5 in the inner volume 3 of the rolling membrane 1 which may be used to guide the rolling membrane 1 along a desired direction as it is rolling out in the distal direction D. The guiding element 5 may include a curved wire which may extend to the outside of the patient and may be manipulated by a physician. The guiding element 5 may be adapted to be or be brought slidably in contact with an inner side of the rolling membrane 1 at a sliding spot 6 that may be arranged at a distal portion of the rolling membrane. The sliding spot may be formed by a tip of the guiding element. A physician may thus steer the rolling membrane 1 by essentially pushing the rolling membrane 1 in a desired direction by the guiding element 5. Steerability may be increased by additionally (partially) inflating and/or deflating the rolling membrane. The rolling membrane 1 is exemplarily located in front of an ostium 7 into which the rolling membrane 1 may expand as it is rolled out along the distal direction D.

FIG. 1b shows the catheter of FIG. 1a in a state in which the rolling membrane 1 has been rolled out along the distal direction D by a certain amount that is smaller than in FIG. 1a (or located more distally than in FIG. 1a). Therefore, the distal end of the rolling membrane 1 has propagated into the ostium region 7 which may be subject to the vascular intervention.

In analogy to FIG. 1b, FIG. 1c shows the catheter of FIGS. 1a and 1b in an even further rolled out state in which the rolling membrane 1 has been rolled out into the ostium 7 even further.

It is emphasized that regardless of the extent to which the rolling membrane 1 has been rolled out, the sliding spot 6, associated with the guiding element 5 may remain at the distal (tip) portion of the rolling membrane 1. The reference numeral 2 is the point at which the retraction element 4 is joined to the first end of the rolling membrane 1. The rolling membrane 1 may be adapted to be rolled out by at least 5 cm, at least 10 cm, at least 20 cm, or at least 30 or 40 cm, for example. For example, it may be rolled from outside the patient, or from a position inside the patient close to the surface of the patient's body to the ostium 7.

FIGS. 2a-2c show a second possible embodiment of a catheter. In this second embodiment, the catheter may be provided with a diameter which is smaller than the diameter of a vessel of a patient into which the catheter is to be inserted. Besides the diameter of the catheter, the catheter may be implemented identically as the catheter described above with reference to FIGS. 1a-1c. In particular, FIGS. 2a-2c show the rolling membrane 1 in different states as it is rolled out into the distal direction D into an ostium 7.

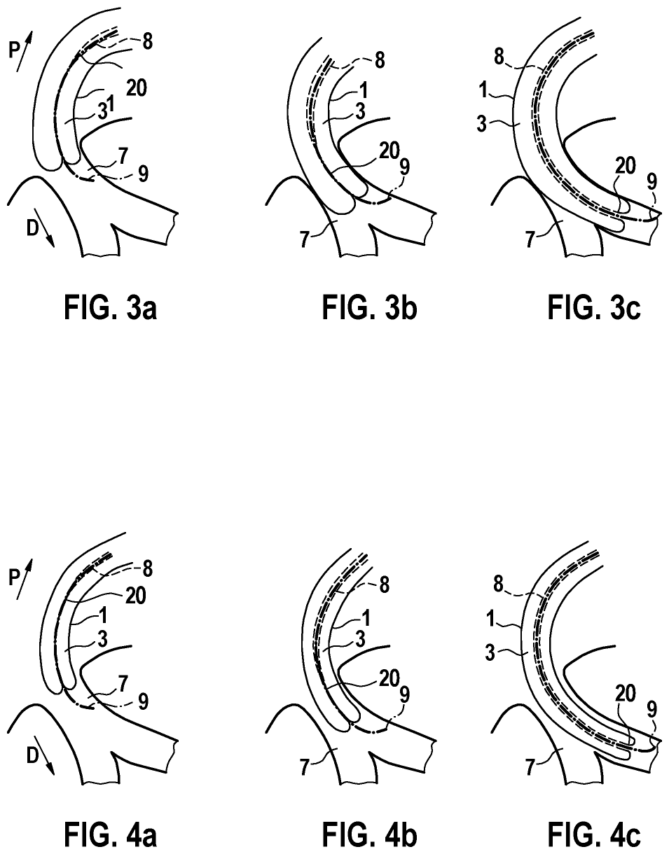

FIGS. 3a-3c show a third embodiment of a catheter. In this third embodiment, the catheter may be provided with a diameter which has the same diameter as the diameter of a vessel of a patient into which the catheter may be inserted.

FIG. 3a shows a catheter, located in an aorta of the patient, including a rolling membrane 1 in an initial state in which at least a portion of the rolling membrane 1 may be folded inwards into itself by a certain amount. The rolling membrane 1 may further include an inner volume 3 identical as outlined above with reference to FIGS. 1 and 2. The reference numeral 20 refers to the point at which the inner shaft 8 is joined to the first end of the rolling membrane 1. The portion of the rolling membrane 1 which is folded inwards by the amount may be attached to an inner shaft 8. The inner shaft 8 may preferably be implemented with a cylindrical shape and the inner shaft 8 may preferably be provided with an inner lumen. The inner lumen 8 may extend to the outside of the body of the patient. In a preferred implementation, the inner shaft 8 may be made from a flexible material such that it may be bendable and may follow the vessel of the patient as it is pushed along the distal direction D along the vessel of the patient.

The catheter according to this third embodiment may include a guidewire 9 which may be located in a center region of the rolling membrane 1 and in the inner shaft 8 (e.g., if the rolling membrane 1 provides sufficient stiffness and if the required guidance angles of the rolling membrane 1 are not too large for the rolling membrane). The guidewire 9 may extend to the outside of the body of the patient and may preferably include a wire (e.g. having a curved tip). The guidewire 9 may provide a guiding for the rolling membrane 1 as it is rolled out along the distal direction D, e.g., into the ostium 7. In general, the guidewire 9 may move faster than the rolling membrane 1 (twice as fast as the rolling membrane) due to the mechanism of operation of the rolling membrane 1. Therefore, the guidewire 9 may be required to be regularly retracted during the propagation of the rolling membrane 1. Under some circumstances, it may be required that the guidewire 9 is provided with a certain extent of stiffness such that it may withstand bending forces arising from the rolling membrane 1 as it is rolled out. If required, the guidewire 9 may be exchanged by the inner shaft 8.

Alternatively, the physician may eliminate the use of the guidewire 9 and may use the inner shaft 8 alone in the rolled out state for steering. In this case the distal end of inner shaft D may be (slightly) curved. In this case, the rolling membrane 1 may partially be deflated, the inner shaft 8 may be pushed forward to the desired location to steer the rolling membrane 1 prior to reinflating the rolling membrane 1 to further roll it out.

FIG. 3b shows the identical catheter of FIG. 3a in a state in which the rolling membrane 1 is at least partially rolled out along the distal direction D.

FIG. 3c shows the identical catheter as depicted in FIGS. 3a and 3b and described with reference thereto in a state in which the rolling membrane 1 is even further rolled out, approximately to a fully rolled out state. It is emphasized that as a result of rolling out the rolling membrane 1, the inner shaft 8 is pulled to a distal end of the rolling membrane 9 thus forming an inner channel, in a centered region, of the rolling membrane 1, that may be accessed from outside the patient.

FIGS. 4a-4c relate to a catheter according to a fourth embodiment. The catheter according to the fourth embodiment may be implemented identically to the catheter according to the third embodiment as described with reference to FIGS. 3a-3c above. However, as a difference, the diameter of the catheter of the fourth embodiment may be smaller than the diameter of the vessel of the patient.

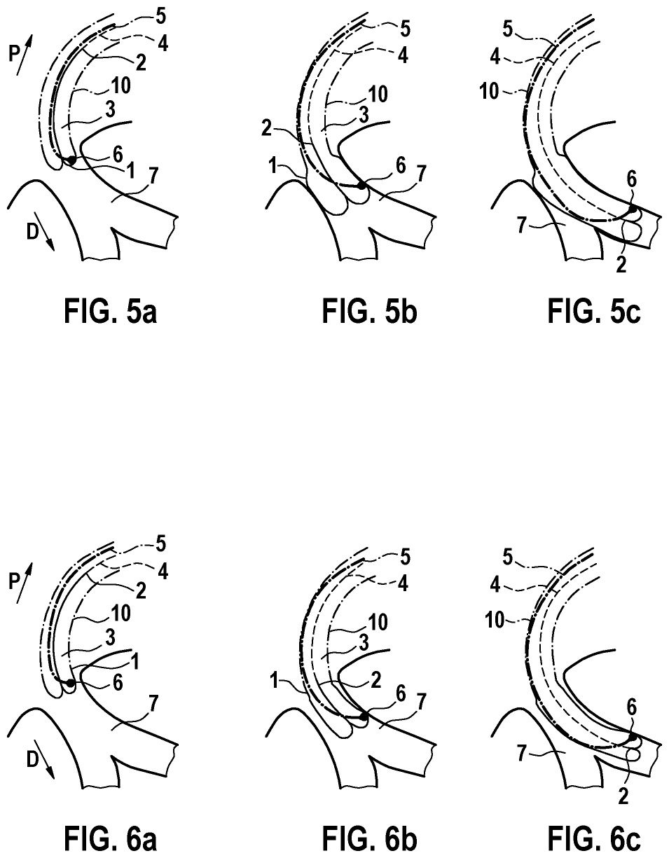

FIGS. 5a-5c depict a catheter according to a fifth embodiment. FIGS. 5a-5c show a rolling membrane, in an initial state (FIG. 5a) and in two further states in which the rolling membrane 1 is at least partially rolled out (FIGS. 5b and 5c) according to the fifth embodiment. In the fifth embodiment, the catheter, and in particular the rolling membrane, may be provided with a diameter with the same size as the diameter of the vessel of the patient.

FIG. 5a shows a catheter including a rolling membrane 1, which may generally be similar to the rolling membrane 1 according to any of the embodiments described with reference to FIGS. 1-4 above. In an initial state, the rolling membrane 1 may be folded inwards by an amount and may form an inner volume 3. The rolling membrane 1, according to the fifth embodiment, may further be provided with a retraction element 4 and a guiding element 5 (located in the inner volume 3 of the rolling membrane 1) which may be slidable arranged at the rolling membrane 1 at sliding spot 6. The reference numeral 2 refers to the point at which the retraction element 4 is joined to the first end of the rolling membrane 1.

However, according to the fifth embodiment, a second (distal) end of the rolling membrane 1 may be attached to an outer shaft 10. The outer shaft 10 may preferably be provided with a cylindrical shape including an inner lumen such as to accommodate the amount of the rolling membrane 1 which is folded inwards. The outer shaft 10 may further also accommodate the retraction element 4 and the guiding element 5. The outer shaft 10 may be made from a bendable and flexible material and may be used to deliver the rolling membrane 1 to a vicinity of an ostium 7 from which the rolling membrane 1 may be rolled out along a distal direction D. As shown in FIG. 5, the rolling membrane 1 is adapted to include a diameter that is larger than that of the outer shaft 10, e.g. a diameter that is approximately equal to a diameter of the vessel into which it is to be inserted, whereas the outer shaft 10 has a smaller diameter than the vessel. In an alternative embodiment, the outer shaft 10 includes or consists of a diameter that is larger than that of the rolling membrane 1, e.g. a diameter that is approximately equal to or smaller than a diameter of the vessel into which it is to be inserted. The distal end of outer shaft D may be (slightly) curved.

FIGS. 6a-6c show a catheter according to a sixth embodiment. The catheter according to the sixth embodiment may be implemented identically to the catheter according to the fifth embodiment as described with reference to FIGS. 5a-5c above. However, the rolling membrane 1 according to the sixth embodiment may be provided with a diameter which smaller than a diameter of the vessel of the patient into which it is to be inserted. The diameter of the rolling membrane may still be larger, e.g. by about 5-500% or 10-50%, than that of outer shaft 10 as shown in FIGS. 6a-6c. In other examples, rolling membrane 1 may include approximately the same diameter as outer shaft 10. In an alternative embodiment, the outer shaft 10 includes or consists of a diameter that is larger than that of the rolling membrane 1, e.g. a diameter that is approximately equal to or smaller than a diameter of the vessel into which it is to be inserted. The distal end of outer shaft D may be (slightly) curved.

FIGS. 6b and 6c show the catheter of FIG. 6a in states in which the rolling membrane 1 has at least partially (FIG. 6b) or almost fully (FIG. 6c) been rolled out.

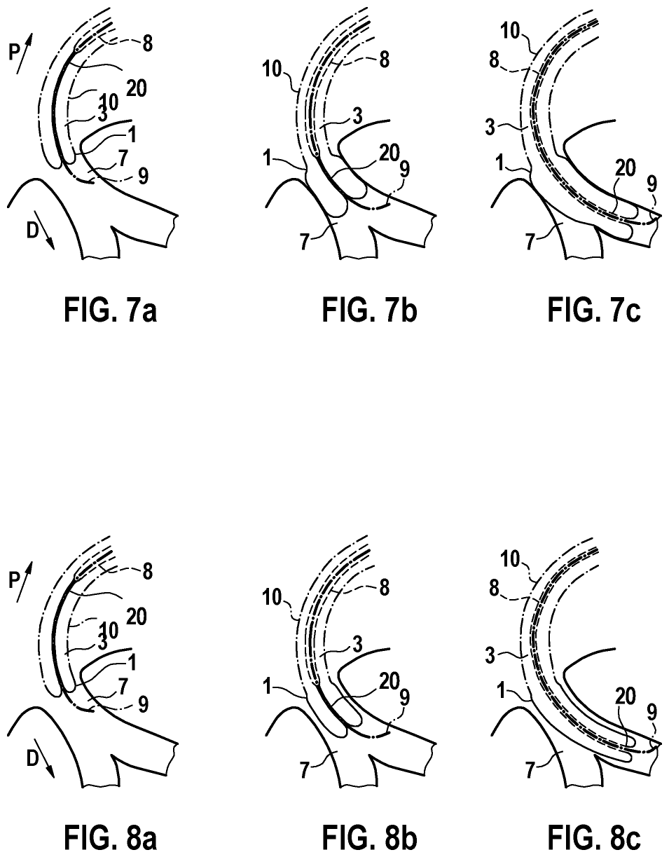

FIGS. 7a-7c show a catheter, including a rolling membrane, in an initial state (FIG. 7a) and in two further states in which the rolling membrane 1 is at least partially or fully rolled out (FIGS. 7b and 7c) according to a seventh embodiment. In the seventh embodiment, the catheter, and in particular the rolling membrane, may be provided with a diameter of the same or similar size as the diameter of the vessel of the patient. Generally, in embodiments, in which the diameter of rolling membrane 1 is similar to the diameter of the vessel of the patient, better anchoring of the rolling membrane 1 to the inner wall of the vessel of the patient may be achieved.

The rolling membrane 1 according to the seventh embodiment may be implemented similarly to the rolling membrane 1 of the embodiments as described above with reference to FIGS. 1-5. The rolling membrane 1 may further be adapted to form an inner volume 3 and may be rolled out along a guidewire 9, wherein the guidewire 9 may be implemented similarly as outlined above with respect to the third and fourth embodiment (FIGS. 3 and 4). In the seventh embodiment, a first end of the rolling membrane 1 (foldable inwards, e.g. by an amount) may be connected to an inner shaft 8. A second end of the rolling membrane 1 may be connected to an outer shaft 10. The reference numeral 20 refers to the point at which the inner shaft 8 is joined to the first end of the rolling membrane 1.

FIGS. 8a-8c show a catheter according to an eighth embodiment. The catheter according to the eighth embodiment may be implemented similarly to the catheter according to the seventh embodiment as described with reference to FIGS. 7a-7c above. However, the rolling membrane 1 according to the seventh embodiment may be provided with a diameter which is smaller than the diameter of the vessel of the patient. In cases in which the diameter of the rolling membrane 1 is smaller than the diameter of the vessel of the patient, an unobstructed blood flow may be achieved during the vascular intervention. The diameter of the rolling membrane 1 may still be larger (e.g. by about 5-500% or 10-50%) than that of outer shaft 10 as shown in FIGS. 8a-8c. In other examples, rolling membrane 1 may include approximately the same diameter as outer shaft 10.

FIGS. 8b and 8c show the catheter of FIG. 8a in which the rolling membrane 1 has further been rolled out. With respect to the aforementioned eight embodiments, it may be summarized that each of the embodiments combines the functionality of a guiding catheter or guidewire with that of a balloon catheter (as the rolling membrane 1 may be expandable in a radial direction). Therefore, the overall complexity of a catheter according to each of the aforementioned embodiments may be reduced. Since only a single catheter is required in each of the above-mentioned embodiments, the procedural time which is necessary to perform a vascular intervention may also be reduced as only a single catheter needs to be inserted into the vascular system of the patient. The rolling membrane 1 may in each of the embodiments preferably be implemented such that the rolling out occurs essentially frictionless (even if the rolling membrane 1 has a similar diameter as the respective vessel).

Moreover, and in view of the above-mentioned embodiments, it may be possible that the rolling membrane 1 may be provided such that it may have a fixed diameter (which is either smaller than the diameter of the vessel or which is identical to the diameter of the vessel, and which may be reached upon full inflation). However, it may also be possible that the diameter of the rolling membrane 1 may be variable to some extent such that the diameter of the rolling membrane 1 may be adapted to the specific requirements of a vascular intervention (e.g. by providing an elastic rolling membrane with relatively low Young's modulus).

In any of the above-mentioned embodiments, in which the catheter includes an outer shaft 10, the outer shaft 10 may be used to support the proximal catheter area as the catheter is pushed through the vascular system of the patient, e.g., to an ostium 7. Moreover, as the rolling membrane 1 may be folded inwards into the outer shaft 10 as it is delivered to the ostium 7, the outer shaft may also provide protection to the rolling membrane 1.

In any of the above-mentioned embodiments, the rolling membrane 1 may be filled with radiopaque solution which may be visible in an angiogram. Visibility may further be improved by one or more x ray markers which may be located either on the inner shaft 8 of the rolling membrane 1 and/or on the surface of the rolling membrane 1 in order to give the physician an indication by which distance the rolling membrane 1 is rolled out. The distance between the one or more x ray markers may be pre-defined. The physician may—based on CT images—determine the location of the stenosis and may thus control the length of rolling membrane 1 by which it needs to be rolled out to arrive at a desired location in a vessel of the patient. Moreover, with the help of the one or more x ray markers it may be ensured that the catheter is adequately rolled out.

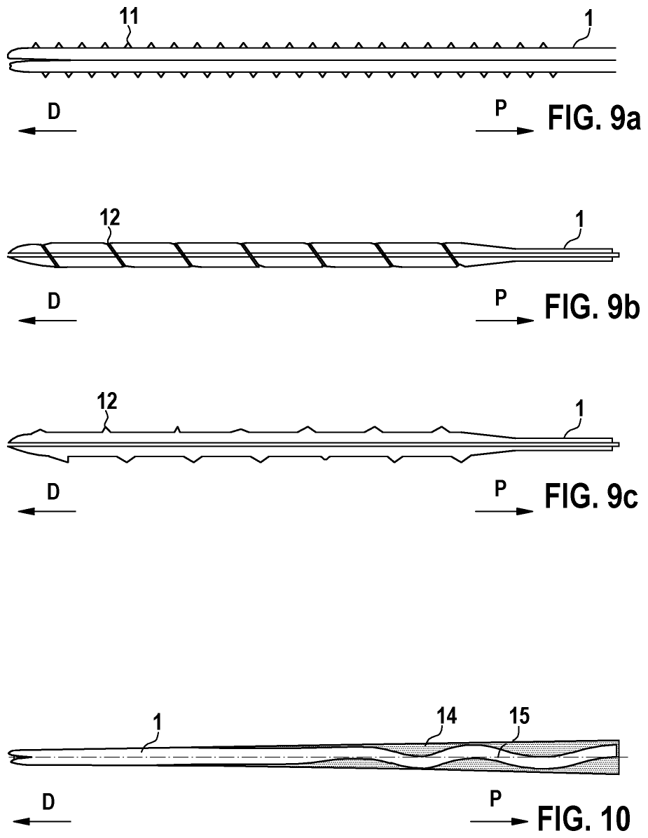

FIGS. 9a-9c show potential aspects of the rolling membrane 1 that may be used independently but also in conjunction with any of the other aspects described herein. The rolling membrane 1 may generally be provided with a uniform/smooth surface or may at least partially be provided with a non-uniform surface, as shown in FIGS. 9a-9c. The usage of a non-uniform surface may in particular provide the advantage of providing at least locally increased friction of the rolling membrane 1 to the inner side of the vessel of the patient.

According to FIG. 9a, the rolling membrane 1 may include one or more (spike-like) protrusions 11 on its surface. The protrusions may be arranged according to a regular pattern and/or may be identical to each other. They may for example be separately fabricated and attached to the membrane.

In a further implementation aspect, the rolling membrane 1 may be thermoformed such that the non-uniform surface of the rolling membrane 1 may be provided with one or more circumferential grooves 12 (FIG. 9b) or with one or more circumferential peaks 13 (FIG. 9c). The protrusions may vary in size and shape. But it is also contemplated that they may be identical to each other. Irrespectively, they may be arranged according to a regular pattern on the surface.

It is noted that non-uniform surfaces of the rolling membrane 1 may be advantageously used as scoring elements. to initiate earlier multiple cracks in the calcified stenosis that propagate when increasing the hydraulic pressure inside the balloon.

FIG. 10 shows a further aspect of the rolling membrane 1 that may be used independently from but also in conjunction with any of the further aspects described herein. The rolling membrane 1 is located in a blood vessel 14 of the patient and may possess a diameter which is smaller than the diameter of the vessel of the patient at least in some regions of the vessel. The rolling membrane 1, may be adapted to acquire a helix-like shape, similar to a corkscrew and/or to twine about, e.g., a central axis 15 of the blood vessel 14. This implementation of the rolling membrane 1 may allow for unobstructed blood flow while ensuring a certain anchoring of the rolling membrane 1 to the inner wall of the blood vessel of the patient.

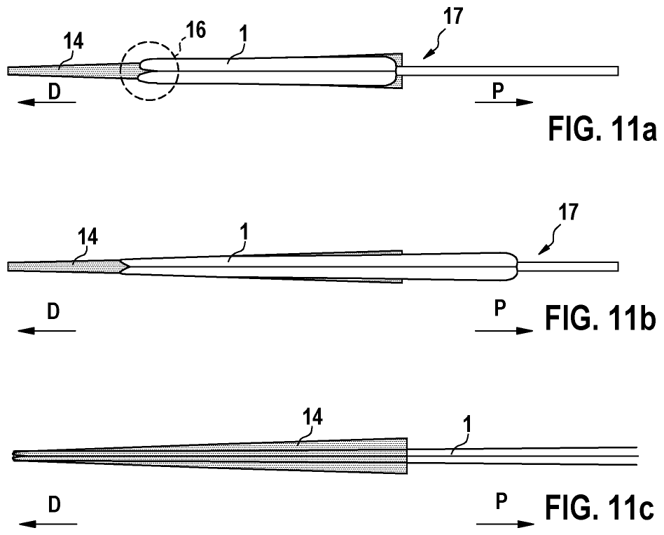

FIGS. 11a-11c show a further implementation aspect of the rolling membrane 1, in accordance with the above-mentioned embodiments and implementation details, wherein the rolling membrane 1 may be adapted to enter a blood vessel 14 which may become narrower along a longitudinal extension of the vessel. The rolling membrane may be adapted to include a tapered diameter as it is rolled out distally. For example, the rolling membrane may have a diameter at its proximal end between 1.5 to 5.0 mm and a diameter on its distal end down to 1.33 mm).

With reference to FIG. 11a, if the rolling membrane 1 arrives at a location 16 in the blood vessel 14 at which the diameter of the blood vessel 14 is too small for the rolling membrane 1 to propagate further into the vessel (such locations 16 may be visible in an angiogram), it may be possible to (at least partially) deflate the rolling membrane 1, the catheter 17 (e.g. by an outer shaft) may be pulled back by a certain distance (FIG. 11b), and the rolling membrane 1 may be rolled out again (FIG. 11c). This deflation/inflation step may allow that the rolling membrane 1 leaves the catheter 17 (and/or the outer shaft 10) by a larger distance and may thus acquire less radial (but increased longitudinal) expansion when being inflated. Therefore, also portions of the blood vessel 14 of the patient, which become narrower with increasing longitudinal distance, may undergo vascular interventions by a catheter according to aspects of the present application. Therefore, a single catheter may be used to access different vessel sizes. It is noted that in FIG. 11, the diameter varies continuously, but it may also be contemplated that the diameter varies in steps, such that portions of the rolling membrane 1 have approximately a constant diameter.

As an alternative to the aspect as described with reference to FIG. 11a above, it may also be possible to provide the rolling membrane 1 with a diameter that increases as the membrane is rolled out distally.

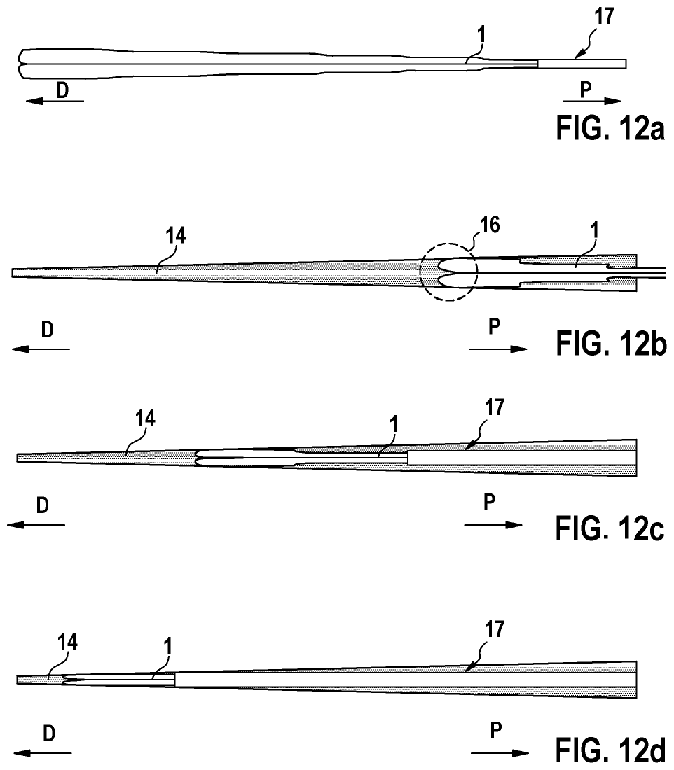

FIGS. 12a-d show a corresponding example with a stepped-diameter. As depicted in FIG. 12a, the rolling membrane 1 may be provided with step-wise increasing diameters from a location from which the rolling membrane 1 is rolled out to a distal-most location of the rolling membrane 1. Also, continuous variations as shown in FIGS. 11a-c are possible. If the rolling membrane 1 arrives at a location 16 in the blood vessel 14 of the patient at which the diameter of the blood vessel 14 is too small for the rolling membrane 1 to further propagate along the distal direction D of the blood vessel, the rolling membrane 1 may at least partially be deflated, the distance by which the rolling membrane 1 was rolled out from the catheter 17 may be decreased and the rolling membrane 1 may be inflated again. Therefore, since the rolling membrane 1 is rolled out by a smaller distance from the catheter 17, the rolling membrane 1 includes a smaller diameter. Therefore, the rolling membrane 1 may access even narrower sections of the blood vessel 14 of the patient. Again, it may be facilitated that only a single catheter is required to access sections with different diameters of the blood vessel 14 of the patient.

The potential special designed implementations of the rolling membrane 1, as described with reference to FIGS. 11 and 12 above, may further allow physicians to determine the anatomical structure of vessel based at least in part on the steps of deflating/inflating and retraction of at least a portion of the rolling membrane 1.

In the following, potential application scenarios will be described in further detail which may rely on one or more embodiments and aspects as outlined above.

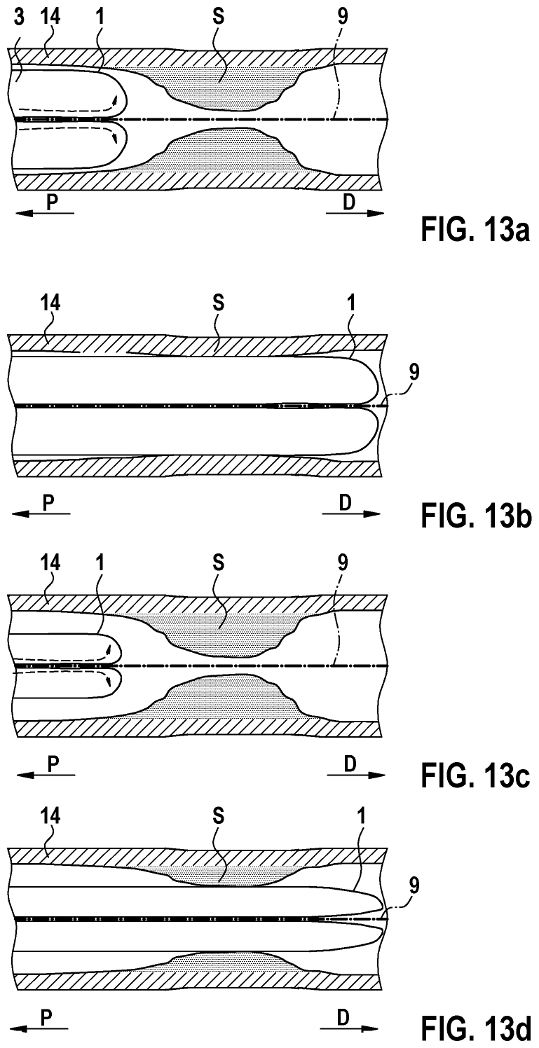

FIGS. 13a-13d show the process of a balloon angioplasty to dilate at least a local stenosis S in a blood vessel 14 of a patient by a catheter according to an embodiment and/or aspect. It is noted that the procedure described below may be performed with a catheter with or without an inner shaft 8 and/or an outer shaft 10.

FIG. 13a depicts a stenosis S in the blood vessel of the patient 14. The rolling membrane 1 (provided with a diameter similar to the diameter of the blood vessel 14 of the patient) has already been rolled out to the vicinity of the stenosis S with the help of the guidewire 9 (which may also be omitted). As the pressure in the inner volume 3 of the rolling membrane 1 is increased, the rolling membrane 1 may further be rolled out and may roll into the constriction, i.e., the stenosis, in the blood vessel 14 of the patient either by itself without any further manipulation by a physician or through an interplay of pressurizing the rolling membrane and/or forward movement and retraction of the guidewire. Since the pressure in the inner volume 3 of the rolling membrane 1 may adapted to the needs of the vascular intervention, the rolling membrane 1 may be capable of dilating the stenosis S even if only a small section of the rolling membrane 1 may be capable of entering the stenosis. The rolling membrane may thus literally roll into the stenosis and at the same time gradually dilate it, as the rolling membrane rolls distally.

FIG. 13b shows an exemplary result of the angioplasty, based on a catheter according to an embodiment of the present invention. In FIG. 13b, the stenosis S has been dilated to a large extent such that the rolling membrane 1 may now be able to roll through the blood vessel 14 without further obstacles (e.g., for further treatments). Since the stenosis S has been dilated to a large extent, unobstructed blood flow in the blood vessel 14 may be facilitated (again).

FIGS. 13c and 13d show the angioplasty of a stenosis S in analogy to the angioplasty described with reference to FIGS. 13a and 13b above. In the exemplary illustration according to FIGS. 13c and 13d, the diameter of the rolling membrane 1 is chosen to be smaller than the diameter of the blood vessel 14. In such scenarios, the rolling membrane 1 may also roll into even narrower lesions and may at least partially dilate a stenosis S (FIG. 13d). It may also be possible that a very narrow lesion may be at least in part be dilated with a rolling membrane 1 provided with a diameter which is smaller than the diameter of the blood vessel 14. In a subsequent step, the rolling membrane 1 may be provided with a diameter (e.g. by a rolling membrane as outlined with reference to FIGS. 11 and 12) which is similar to the diameter of the vessel 14 such as to fully dilate the stenosis S as described with reference to FIGS. 13a and 13b above.

To summarize, even the passage and dilation of very narrow lesions may be facilitated due to the hydraulically assisted and friction-free rolling capability of the rolling membrane 1. Moreover, the balloon length, i.e., the length of the radially inflated portion of the rolling membrane 1 may be variable according to the needs of the vascular intervention.

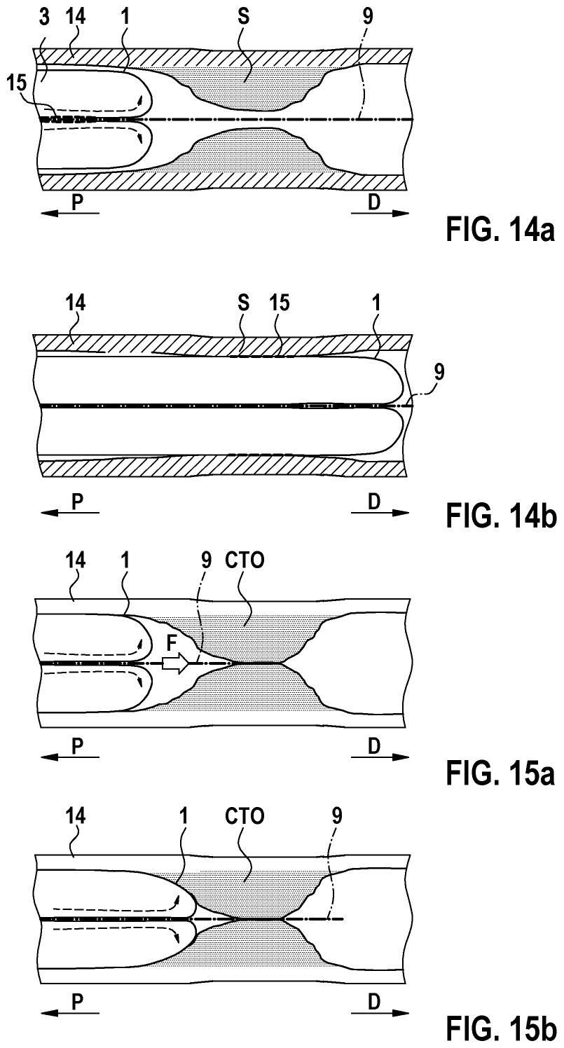

FIGS. 14a and 14b show a potential application of a catheter in which the rolling membrane 1 may adapted to deliver a drug 15 to a distal-most spot of the rolling membrane 1 in accordance with any of the above-mentioned embodiments. It is noted, that the procedure described below may be performed with a catheter with or without an inner shaft 8 and/or an outer shaft 10.

FIG. 14a depicts a rolling membrane 1 which has been rolled out into the vicinity of a stenosis S, e.g., along a guidewire 9 (which may also be omitted). The rolling membrane 1 may, at one or more locations, be coated with a drug 15. The location, at which the rolling membrane 1 may be coated with a drug 15 may preferably be chosen such that the location is protected by an inwards folded portion of the rolling membrane 1 and such that the drug 15 may only be released in the vicinity of a lesion, e.g., the stenosis S. Therefore, the drug 15 may be delivered to the location at which it may be useful and may not be diluted by the blood flow, e.g. as shown in FIG. 14b. FIG. 14b shows a situation in which the rolling membrane 1 has been rolled out such that drug 15 has been folded outwards such that it is located adjacent to the side walls of blood 14 at which a stenosis S is or may have been present. A suitable drug may, e.g., be a drug which may prevent a restenosis or a drug which may dissolve a thrombus (i.e. anti-thrombotic drugs). Suitable drugs may be anti-proliferation drugs e.g. Paclitaxel or Sirolimus drugs. In some applications, it may be possible that a lesion (e.g., the stenosis S) may be treated with the drug 15 prior to being treated by a mechanical dilution by a catheter as described herein (e.g. the drug may be delivered by the rolling membrane 1 in a low pressure state and then the rolling membrane 1 may be pressurized). In some applications, it may also be possible that a lesion (e.g., the stenosis S) is mechanically dilated first and in a subsequent step, further treated by the drug 15, which may directly be delivered to the location of the stenosis. Since the length by which the rolling membrane 1 has been rolled out essentially determines the location at which the drug 15 may be folded outwards (and thus released), the drug impact range may be made adjustable. Therefore, a drug coated balloon catheter may be provided which may still propagate to a certain location in the blood vessel 14 of the patient by itself. A knob/stopper on the handle of the rolling membrane catheter can be used to limit the maximum longitudinal movement of the inner shaft which again controls the evertable length of the rolling membrane.

FIGS. 15a-15b show a further application of a catheter which may be used for crossing a chronic total occlusion (CTO) of a blood vessel in accordance with the embodiments as described herein.

FIG. 15a shows an exemplary situation in which the rolling membrane 1 has been rolled out into the vicinity of a CTO, i.e., total occlusion of a blood vessel 14.

In some implementations, the rolling membrane 1 may be provided with a surface which may be adapted to apply a force to, e.g., a guidewire 9 (which may be located in a center portion of the rolling membrane 1). As a result of folding the rolling membrane 1 further outwards (e.g., as a result of pressurizing the inner volume 3 of the rolling membrane 1), a potential force transfer from the rolling membrane 1 onto at least a portion of the guidewire 9, which is located in the center region of the rolling membrane 1, may be facilitated. Therefore, the guidewire 9 may experience a longitudinal pushing force along a distal direction D. If the pressure applied to the inner volume 3 of the rolling membrane is sufficient and the friction between the guidewire 9 and a surface section of the rolling membrane strong enough, even a CTO may be crossed as a result of the hydraulically assisted pushing force F and any buckling back of the guidewire 9 may be prevented. It is further noted that it may be preferable that the rolling membrane 1 has sufficient anchoring to the inner wall of the blood vessel 14 of the patient to be able to apply sufficient force onto the guidewire 9. This may be facilitated by further aspects as described herein.

FIG. 15b relates to FIG. 15a and shows the CTO at a later stage in which the CTO has successfully been crossed by the guidewire 9.

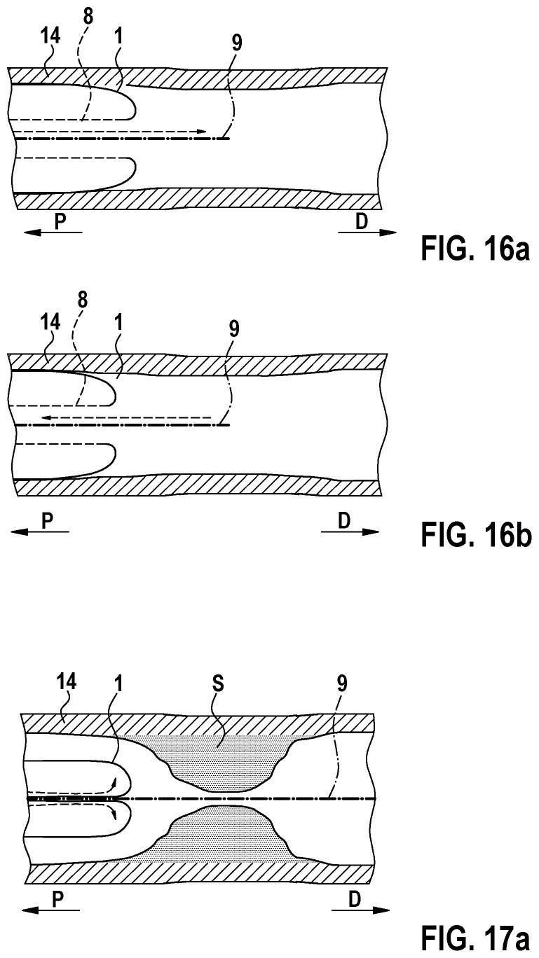

FIGS. 16a-16b show a further application in which the catheter may include at least an inner shaft 8 and which may be in accordance with the respective embodiments, which relate to an inner shaft, as outlined above. By providing a catheter with an inner shaft 8, to which an initially inwards folded amount (not depicted in FIG. 16) of the rolling membrane 1 may be connected, a fluid delivery system may be facilitated as depicted in FIG. 16a. The rolling membrane 1 may be rolled out such that a distal end of the inner shaft 8 may be pulled to the distal-most location of the rolling membrane 1. In a preferred application, the rolling membrane 1 may be positioned (and preferably anchored) at a location at which a certain fluid has to be released. Since the inner shaft 8 may provide a lumen that extends to the outside of the body of the patient, a physician may insert a certain fluid (e.g., a drug, a contrast medium) into the inner shaft which may then be transported and released at the distal-most location of the catheter (the rolling membrane 1).

In applications in which the rolling membrane 1 has the same diameter as the blood vessel 14 of the patient, it may be possible to avoid any dilution of the released drug by the blood flow such that the medical effect of the released drug may be increased. A further advantageous effect may be seen that the overall dose of a drug may be reduced as the drug may be released directly at the location of interest (e.g., the vicinity to a lesion such that a “local therapy” may be facilitated). Potential applications of such a drug delivery system may, e.g., be also seen in cancer treatment, distal lysis treatment for T-segment elevation myocardial infarction (STEMI), etc. The same also applies to the exemplary usage of a contrast medium which may reduce the potential health risks for the patient.

In some application scenarios, and as depicted in FIG. 16b, the inner shaft 8 may also be used to aspirate certain objects (e.g. by applying a negative pressure to the inner lumen of the inner shaft 8), located in close-vicinity of the distal-most part of the rolling membrane 1. Objects of interest to be aspirated may, e.g., be (soft) thromboses and/or debris arising from an atherectomy procedure or any other type of procedures. The same may apply to remnants of drugs and contrast mediums, etc.

Although a guidewire 9 is shown in FIGS. 16a-16b, it may also be omitted.

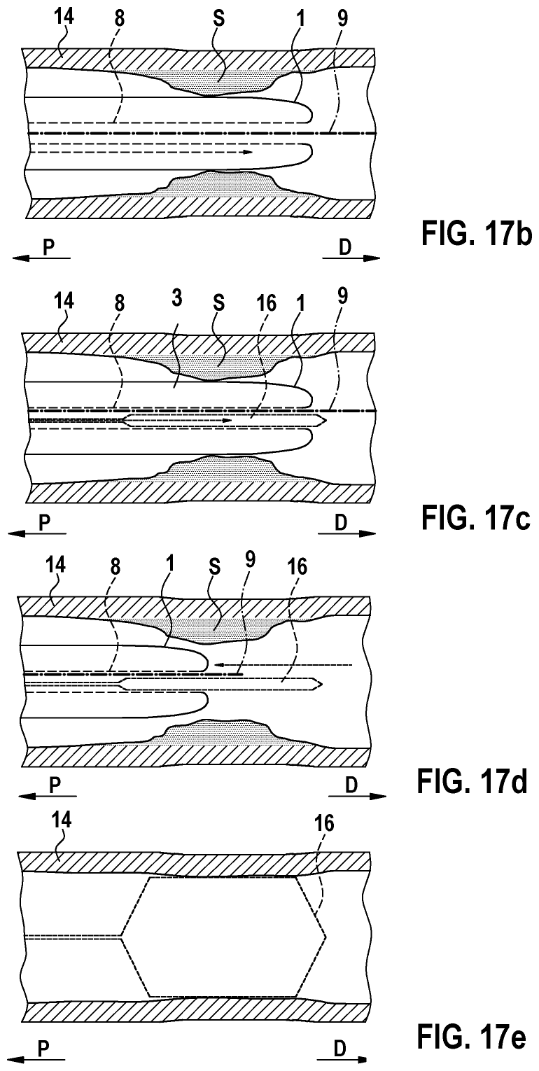

FIGS. 17a-17e show a further application of a catheter, in accordance with any of the above-mentioned embodiments, in which the catheter may be used as a device delivery system.

FIG. 17a shows an exemplary application of a catheter in which a rolling membrane 1 has been rolled out, e.g., by an optional guidewire 9, into the vicinity of a stenosis S. The rolling membrane 1 may further be rolled out into the stenosis S (FIG. 17b) and may be inflated such that the stenosis S may at least partially be dilated (as described above, e.g., with reference to FIG. 13. In some applications, it may be advantageous that the rolling membrane 1 is fully rolled out such that an inner shaft 8 may be pulled to the distal-most part of the rolling membrane 1. In view of a subsequent delivery of a (functional, e.g., medical) device 16 (as further described below), this procedure may be understood as a pre-dilation, followed by a potential full dilation of the stenosis, e.g., at least based in part on the delivered device 16.

In some applications, the device 16 may be inserted into the inner shaft 8 and pushed forward to the distal-most part of the rolling membrane 1, as depicted in FIG. 17c. The required pushing force may be applied from outside the body of the patient, e.g., by a physician. In some scenarios, the device 16 may be held in the center of the rolling membrane 1 as a result of the pressurization of the inner volume 3 of the rolling membrane 1. It may thus be required to deflate the rolling membrane (FIG. 17d) by such a pressure difference that the device 16 may further be pushed out from the rolling membrane. For placing the device 16 at the location of the stenosis S, it may also be required to retract the rolling membrane 1 by a certain distance. As a result, the device 16 may be unloaded from the catheter and the rolling membrane 1 and may thus be placed at a certain location for, e.g., a subsequent treatment (such as to dilate the stenosis S as illustrated in FIG. 17e). As an example, the device 16 may be a simple balloon catheter (which may only include a tube and a balloon) which may, e.g., fully dilate the stenosis S. In some cases, the respective balloon catheter may include an own shaft which may extend to the outside of the body of the patient such that the balloon catheter may be (hydrodynamically) be inflated. This procedure for delivering the device 16 may also be referred to as the flexible type of device delivery.

Various devices 16 may be delivered via the inner shaft 8 of the rolling membrane 1. Some of the examples of devices 16 which may be delivered via the inner shaft 8 are any sort of catheters, such as, e.g., simple balloon catheters, standard balloon catheters, high pressure balloon catheters, etc. The delivery of balloon catheters may in particular be relevant if a certain diameter of the balloon catheter is required. Moreover, also bail out devices, embolic protection devices, scoring or cutting elements, self-expandable stents, guidewires, coils, flow diverters, etc., may be delivered. In some applications, it may also be possible to deliver a device 16 such as a camera for endoscopy or to deliver biopsy needles. By a catheter in accordance with an embodiment described herein, it may be facilitated to deliver and/or remove devices 16 from a certain location in the blood vessel 14 of the patient with only a single catheter. In some applications, it may also be possible that more than one device is delivered to a certain location in the blood vessel 14 of the patient, e.g., in subsequent steps. In some cases, it may be possible to spontaneously deliver the device 16 to a lesion, e.g., if it unexpectedly turns out during a vascular intervention that an additional high pressure balloon catheter may be required. This may simplify the overall PCI or (percutaneous ventricular intervention) PVI or neurovascular procedures and may make them less time consuming.

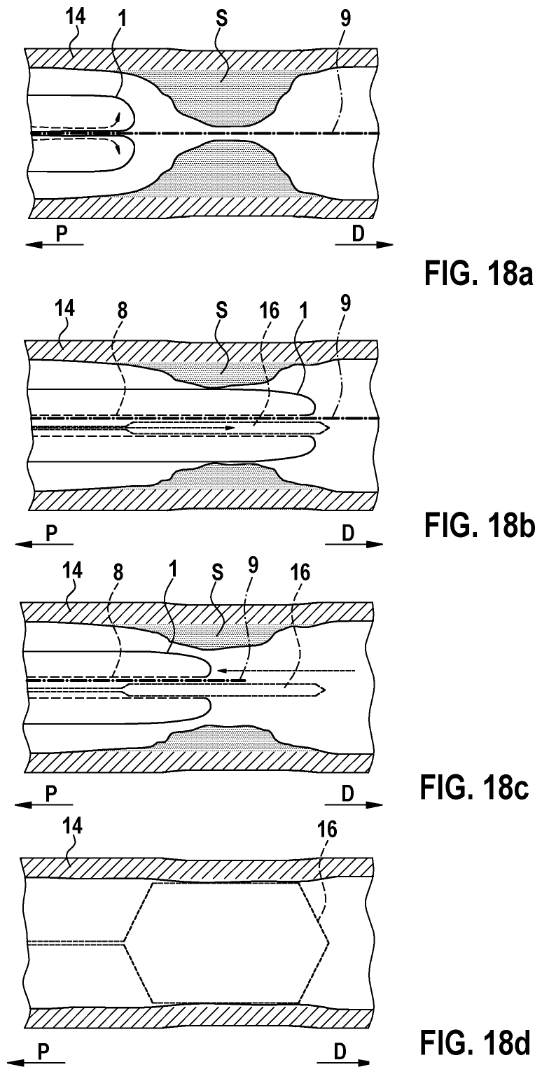

FIGS. 18a-18d show an alternative device delivery procedure during which a device 16 to be delivered may be preloaded into an inner shaft or a center region of a rolling membrane 1. The rolling membrane 1 is provided with a smaller outer diameter as the inner diameter of the vessel 14 of the patient. The delivery steps depicted in FIGS. 18a-18d may be understood in analogy to FIGS. 17a-17e as described above. In particular, the device 16 may be identical to one or more of the devices exemplarily described above with reference to FIGS. 17a-17e.

However, deviating from the delivery process described with reference to FIGS. 17a-17e above, it may be possible that the device 16 may be preloaded into the inner shaft 18. In other words, it may also be possible that the device 16 may not be pushed through the inner shaft 8 to the distal-most part of the rolling membrane 1, but that the preloaded device 16 is essentially pulled to the distal-most part of the rolling membrane 1 as a result of the rolling out of the rolling membrane.

It may also be possible that the catheter does not include an inner shaft 8. In such a case, the device 16 may be preloaded into a center region of the rolling membrane 1 such that the device 16 is encompassed by the inwards folded part of the rolling membrane 1 in a center of the rolling membrane. If sufficient friction arises between the device 16 and the inwards folded part of the rolling membrane 1, the device may be pulled to the distal-most part of the rolling membrane 1 as a result of rolling out the membrane. This delivery method is also referred to as a preload mode. It is further noted that also application may be possible in which the rolling membrane 1 is not attached to an inner shaft (as the catheter has only a rolling membrane without an inner shaft). In such a case, the device 16 to be delivered may be placed in a center region of the rolling membrane which may be formed by the inwards folded portion of the rolling membrane.

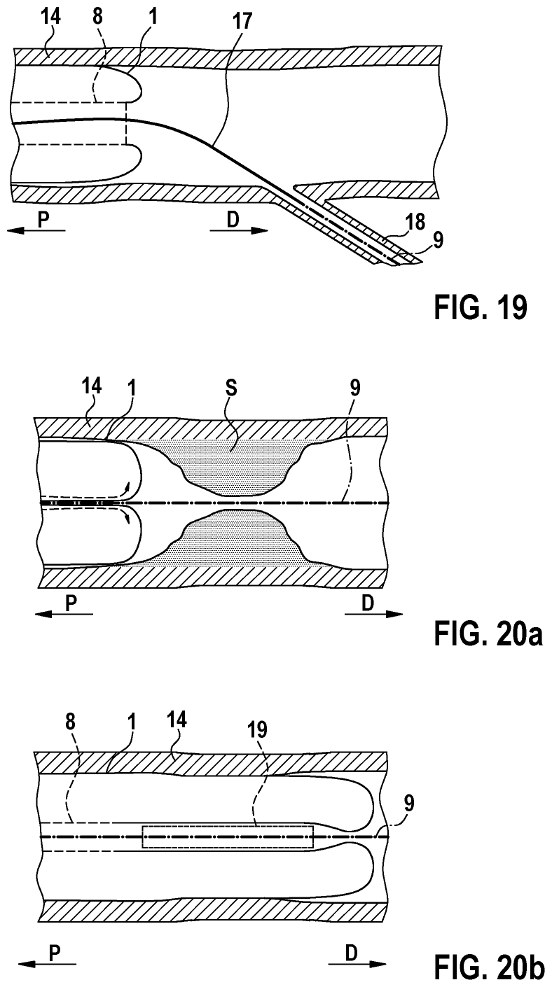

FIG. 19 shows a further application scenario for a catheter, wherein the catheter may be used to deliver a microcatheter 17 to a certain location during a vascular intervention. The catheter described in the following may be understood in accordance with an embodiment as described herein wherein the catheter is provided with an inner shaft 8. FIG. 19 shows a scenario in which the rolling membrane 1 is (fully) rolled out such that the inner shaft 8 has been pulled to the distal-most end of the rolling membrane 1. It may then be possible to push a second, smaller catheter, e.g., a microcatheter 17 through the inner shaft 8 (mother-child technique) and out of the rolling membrane 1 at a distal-most location of the rolling membrane 1. Such an application may allow that the microcatheter 17 may be delivered to a branch of the vascular system of the patient at which a smaller vessel 18 forks from a main vessel 14 wherein the smaller vessel 18 may not be accessible by the rolling membrane 1. The microcatheter may, e.g., be guided into the smaller vessel 18 by a guidewire 9 which may extend through both the microcatheter 17 and the rolling membrane 1. Therefore, also smaller bifurcating vessels 18 may be accessed with a catheter. Since the microcatheter 17 may be pushed through the inner shaft 8 it may not be required to push to different catheters through the vascular system of the patient but to use the “larger” main catheter (including rolling membrane 1) as a delivery system for the microcatheter 17. Moreover, the microcatheter 17 may also be protected by the main catheter as it is pushed through the inner shaft 8 to the position of the bifurcation from which the smaller vessel 18 forks. The rolling membrane may also provide support for the microcatheter 17 close to its intended deployment position. In some further applications, it may also be possible to use the microcatheter 17 as a dedicated delivery system for drugs which may thus be delivered the small vessel 17. This can be particularly advantageous for STEMI patients.

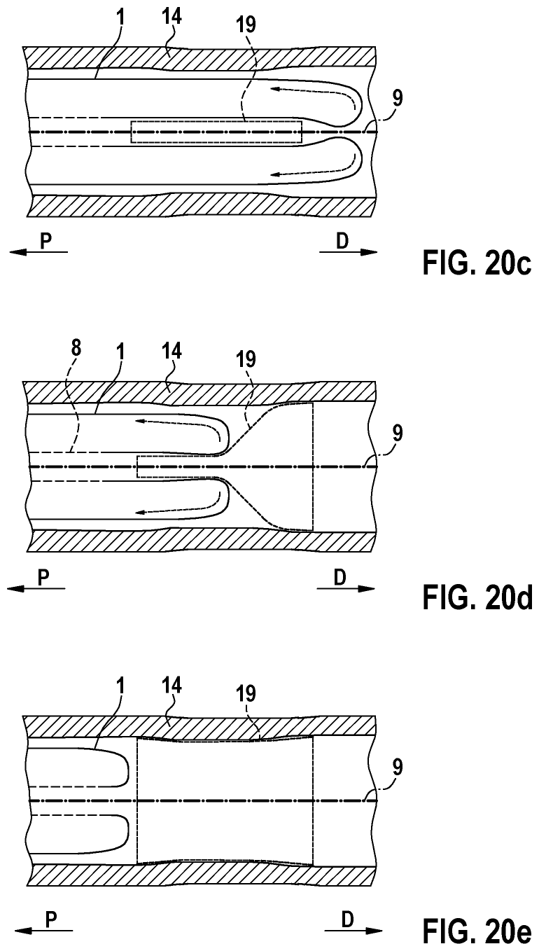

FIGS. 20a-20e show a further application of a catheter according an embodiment in which the catheter may further be used as a delivery system for a self-expandable stent (SX-stent) 19.

FIG. 20a shows an initial situation which may be identical to the situation as described with reference to FIG. 17a above, however, the rolling membrane 1 may be provided with a same or smaller outer diameter as the inner diameter of the vessel 14 of the patient. The rolling membrane 1 may be attached to an inner shaft 8. Moreover, an SX-stent 19 may be preloaded into a centered region of the rolling membrane 1 which may be formed as a result of the inwards folded portion of the rolling membrane in a, not yet fully expanded state of the rolling membrane 1. The rolling membrane 1 may further roll into the stenosis S and may be expanded therein such as to at least partially dilate the stenosis S (FIG. 20b). As a result of further rolling out the rolling membrane 1 (e.g., to dilate the stenosis S), the SX-stent 19 may be pulled to a distal part of the rolling membrane 1. It may further be required to deflate the rolling membrane by certain amount of pressure (FIG. 20c) such that the SX-stent 19 may easily be released and delivered to the distal-most portion of the rolling membrane 1. As soon as the hydraulic pressure in the inner volume of the rolling membrane 1 drops, the SX-stent 19 will expand the inner side of the rolling membrane 1 towards the outer side of the rolling membrane 1 and/or the vessel 14 and friction may stop any further motion. It may also be required to retract the rolling membrane 1 by a certain distance such as to place the SX-stent 19 at the desired location in the vessel 14 of the patient. Then it is necessary to inflate the rolling membrane 1 above a pressure that causes the SX-stent to collapse. Then the retraction of the rolling membrane 1 is possible. The SX-stent 19 may then partially be unloaded from the rolling membrane and may begin to expanding itself (FIG. 20d). As soon as the SX-stent 19 is fully expanded (FIG. 20e) by further retracting the rolling membrane 1, the vessel 14 of the patient may be kept open/unblocked at least by the SX-stent 19. In some examples, the rolling membrane 1 may be adapted to apply a compressive force to the portion of the SX-stent 19 that is still encompassed by the membrane such that a sudden ejection of the SX-stent 19 in a distal direction due to the expansion is avoided.

It is further noted that also applications may be possible in which the SX-stent 19 may be delivered by the rolling membrane 1 without the presence of the inner shaft 8.

This application example shows that the catheter according to an embodiment as described above may be used to deliver an SX-stent 19 to a desired location with a single catheter and minimize procedural time. Moreover, the special frictionless design of the rolling membrane 1 ensures a frictionless SX stent release.

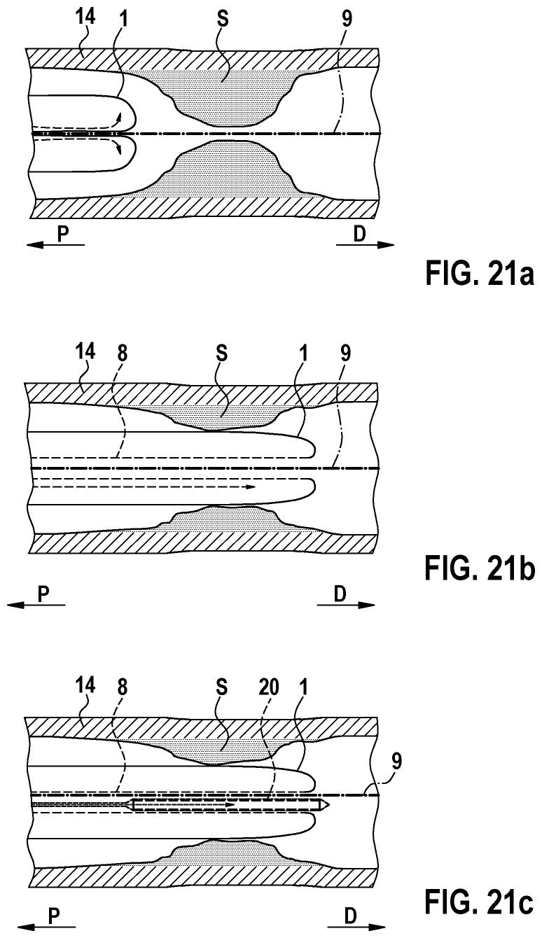

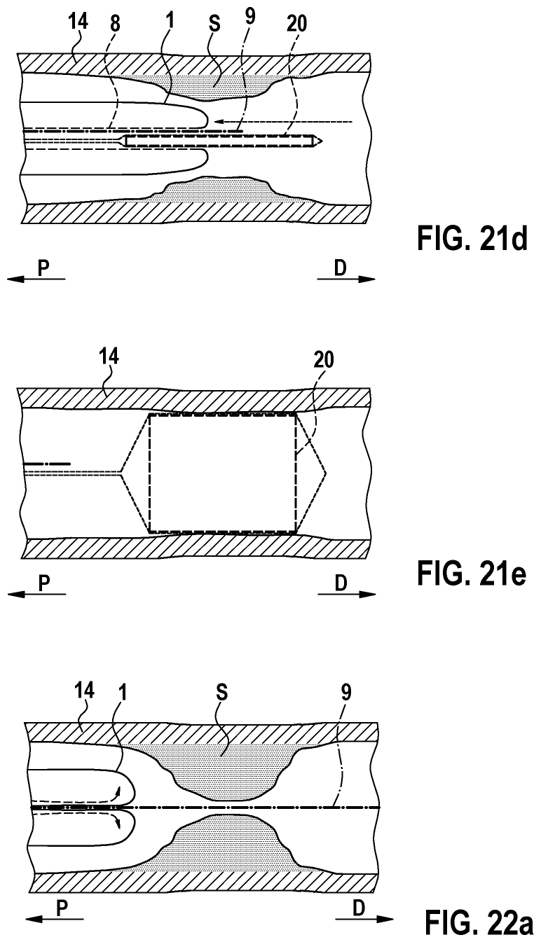

FIGS. 21a-21e show a further application scenario of a catheter according to an embodiment, wherein the catheter may be used to deliver a balloon expandable stent (BE-stent) to a desired location in a blood vessel 14 of a patient. The application scenario described below may be understood in analogy to the delivery procedure described with reference to FIG. 17 above and is thus only be briefly addressed below.

FIG. 21a shows an initial situation identical to the situation described with reference to FIG. 20a above, however, the BE-stent 20 may not be preloaded. The rolling membrane 1 may be rolled into a stenosis S (FIG. 21b), inflated and may thus at least partially dilate the stenosis S. As a result of rolling the rolling membrane 1 (fully) out, the inner shaft 8 may be pulled to the distal-most part of the rolling membrane such that a lumen through the catheter may be formed. It may then be possible to push the BE-stent 20 through the lumen until it reaches the distal-most part of the rolling membrane 1 (FIG. 21c). It may under some circumstances be required to retract and/or deflate the rolling membrane by a certain distance in a proximal direction such that the BE-stent 20 may be placed at a location of the stenosis (FIG. 21d). The BE-stent 20 may then be inflated from outside of the body of the patient by a respective shaft which provides a lumen from the BE-stent 20 to the outside of the body of the patient (FIG. 21e). The BE-stent 20 may then fully dilate a potential stenosis at the location at which it is placed and inflated.

It is noted that in an alternative embodiment the inner shaft 8 may not necessarily required for delivering the BE-stent 20. It may also be possible that the BE-stent 20 may be pushed through a center lumen of the rolling membrane if the rolling membrane opens completely after eversion.

Such a delivery system may provide similar advantages as described with reference to FIG. 17 above.

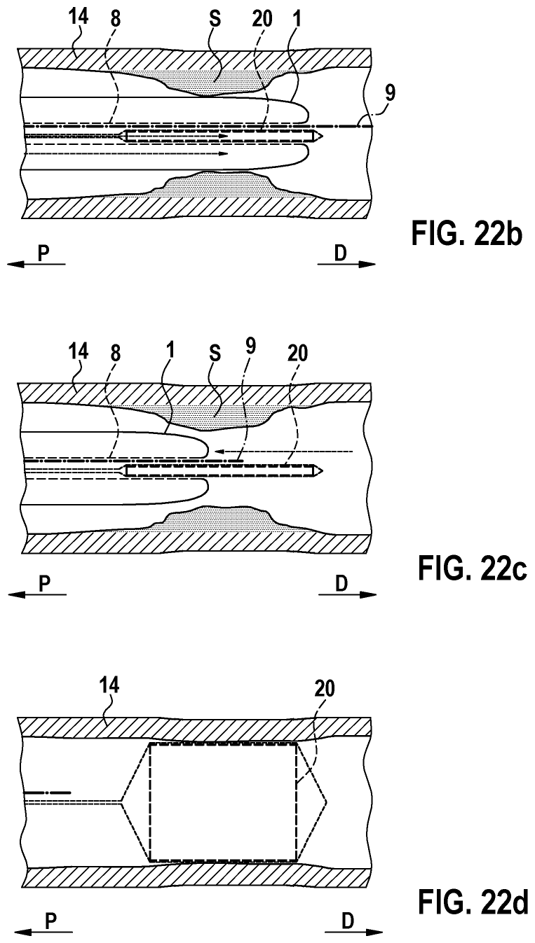

FIGS. 22a-22d relates to an application scenario of a catheter according to an embodiment in which the catheter may be preloaded with a BE-stent 20. The procedure of delivering a BE-stent 20 in preloading mode may include similar procedural steps as outlined above with respect to FIG. 17 with the exception that a preloaded BE-stent 20 is delivered as compared to a (simple) balloon catheter 16. The advantages of such an application of the catheter according to an embodiment of the invention may thus be similar to the advantages outlined with respect to FIG. 17 above.

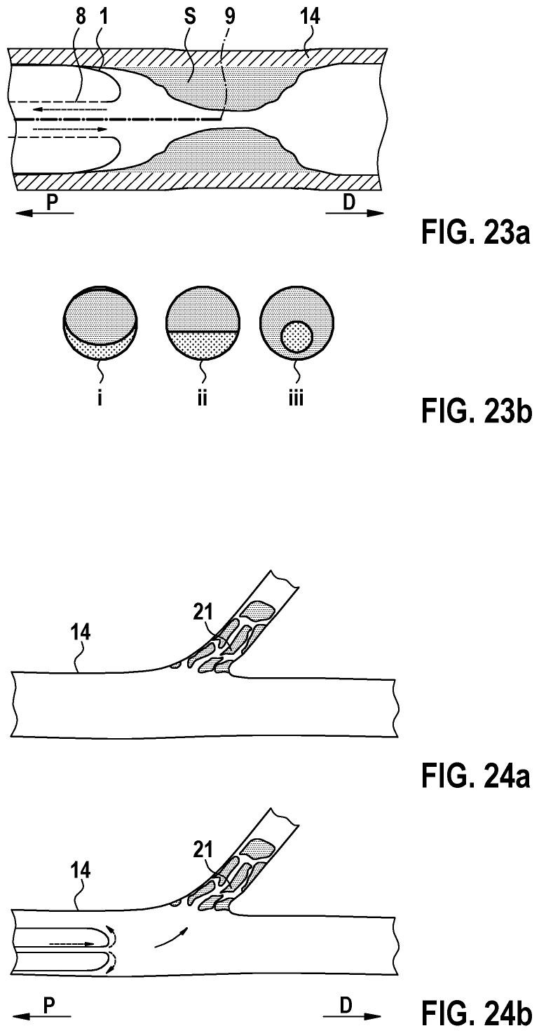

FIGS. 23a and 23b show a further embodiment of a catheter in which a rolling membrane 1 is attached to an inner shaft 8 and may be suitable for atherectomy.

FIG. 23a shows an exemplary situation in which the rolling membrane 1 is (fully) rolled out into the vicinity of a stenosis S. Since the rolling membrane is fully rolled out, the inner shaft 8 has been pulled to the distal-most part of the rolling membrane 1 thus forming an inner lumen through the catheter.

The thus formed inner lumen may extend to the outside of the patient such that a fluid may be inserted to the inner lumen which may be delivered to the distal-most part of the rolling membrane 1 (similarly as described above with reference to FIG. 16). In addition to the delivery possibilities as described with reference to FIG. 16, it may also be possible to guide a water jet through the inner lumen of the rolling membrane 1. A water jet may be understood as a pressurized “beam” of water which may be limited to a defined cross-sectional area. Such a waterjet may allow an atherectomy with the catheter. By the water jet it may be possible to (fully) open the stenosis S by the sole impact of the water jet onto the stenosis S. In some scenarios it may be required to pre-deliver a certain drug to the stenosis S prior to applying the water jet. Additionally, or alternatively it may be possible to deliver a certain drug to the stenosis after applying the water jet onto the stenosis S.

In some scenarios, the catheter may include a dual lumen wherein the inner lumen of the rolling membrane 1 may be divided in to two separate lumina such that a portion of the inner lumen may be used for supplying a certain fluid to the stenosis S whereas the respective remaining portion of the inner lumen may be used for aspiring fluids and/or solids (e.g., debris) from a vicinity of the stenosis S. In such an implementation it may be possible to deliver a certain drug to the stenosis S (which may, e.g., dissolve the stenosis S). Subsequent to the drug delivery, it may be possible to aspire the debris of stenosis and/or potential remnants of the drug supplied beforehand.

FIG. 23b shows three separate arrangements of potential embodiments of a dual lumen. It may thus be possible that a major cross-sectional area (i) of the inner lumen may be provided for aspiring (as thromboses and/or debris may be comparably large particles). In some circumstances, it may be favorable that the cross-sectional areas of the lumina of the dual lumen approximately have similar cross-sectional area (ii) whereas in another embodiment, the area provided for the fluid supply, may be small as compared to the cross-sectional area provided for aspiring and not arranged at a rim of the area provided for the aspiration (iii).

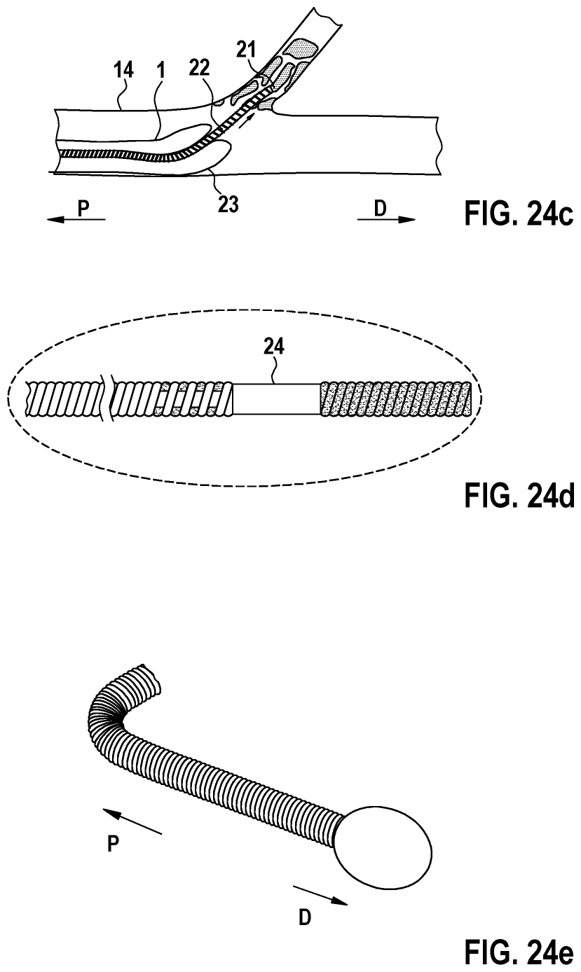

FIGS. 24a-24e show an alternative steering design for steering a rolling membrane 1 in a certain direction as it is rolled out.

FIGS. 24a and 24b show an exemplary situation in which the rolling membrane 1 may propagate through a vessel 14 of a patient. It may further be desired that the rolling membrane 1 should enter a bifurcating vessel 21 (it is noted that the application is not only limited to bifurcating vessels but may also be used for other applications which require small bending radii) which may for example require a small bending radius (as compared to typical bending radii in an aorta) to enter the bifurcating vessel 21. Such small bending radii may not always be possible by a guiding element 5 or a guidewire 9 as described above. In contrast, a steering technique is required which may be capable of allowing smaller bending radii.

FIG. 24c shows an example for a steering technique for the rolling membrane 1 which may allow even smaller bending radii. The steering may be based on a specialized curved wire 22 that may generally serve as a guidewire but be provided with a surface that may cause high friction with the rolling membrane 1. The steering of the rolling membrane in a desired direction may be based on an interplay of pressurizing the rolling membrane and/or forward movement and retraction of the guidewire. Additionally or alternatively, the curved wire 22 may have regions with different surface roughness or regions of different surface materials in order to cause uneven/non-even friction at the front of the rolling membrane 1. Additionally or alternatively, the tip portion of the specialized guidewire 22 may possess a shape with an increased diameter portion (e.g., as depicted in FIG. 24e). In order to interact in an optimized way with the rolling membrane it is supposed that the center region of bended section of the guidewire can be clearly visualized by the used angiographical technique. Since it is acceptable that the guidewire is stiffer within this area this may be achieved by local material amount or the use of stronger x-ray extenuating materials. For steering, the high friction portion of the specialized curved wire 22 may be positioned at a distal portion of the rolling membrane 1 to cause a respective radially uneven (e.g. one sided) friction at the front of the rolling membrane 1 due to the curved design of this the specialized guidewire 22 and/or the tip portion. For steering and the elaboration of an adequate friction of the tip portion of the specialized guidewire 22, the rolling membrane 1 may be deflated completely to enable to manually guide the specialized curved wire 22 in a desired direction. In a subsequent step, the rolling membrane 1 may be inflated again in order to get in contact with the high friction portion of the specialized curved wire 22 as the rolling membrane 1 inflates and rolls out again. Since high friction is provided, the membrane may tend to roll out more at the lower side of FIG. 24c), e.g. at position 23, compared to the upper side of FIG. 24c), hence increasing the extent to which the curved wire 22 is bent. As a result, the rolling membrane 1 may be used to obtain lower bending radii. A similar result may be achieved when using the tip portion, e.g. as shown in FIG. 24e) against which the rolling membrane is to roll out. Additionally, the movement of the specialized guidewire 22 may need to be slowed down in respect to the movement of the rolling membrane 1 in order to cause the required friction.

Additionally, at least a portion of the distal end of the rolling membrane 1 may preferably be made from a viscoelastic material. The radially uneven friction as described above may then cause the rolling membrane 1 to limit radial tension of the balloon of the inflated and pressurized rolling membrane 1 on the inside of the curve. Depending on the properties of the rolling membrane 1 with respect to the pressure dependent length growth of the rolling membrane 1 due to its preferable viscoelastic properties, an at least partial plastic deformation of the distal portion of the rolling membrane 1 at elevated pressure may be facilitated at a location 23. Said deformation may not only cause a curved motion of the rolling membrane, but the rolling membrane 1 may also be plastically formed in a direction of the lesion. In other words, by applying a non-even friction (thus a non-even force) onto a viscoelastic portion of an inner side of the rolling membrane 1 as it rolls out (i.e. as it is being inflated), the portion of the rolling membrane 1 onto which the non-even force acts may be hindered to further expand in e.g. a longitudinal direction. However, due to the preferred viscoelastic properties of the rolling membrane 1, a viscoelastic deformation of the rolling membrane 1 may occur which leads to a change in direction along which the rolling membrane 1 further expands. In this case the area that is less strongly affected by the increased friction in the rolling motion will expand even more (also plastically) and the curved motion of the rolling membrane 1 will thus be intensified. This makes the steering easier for certain lesions and enables a directed force application towards the lesion. The steerability may further be improved by lowering the pressure in the rolling membrane 1. Moreover, under some circumstances the specialized guidewire 22 may additionally be provided with one or more x ray markers for better visibility of the specialized guidewire during angiography.

FIG. 24d shows a detailed view of the specialized (guide) wire 22. The specialized guidewire 22 may include a region 24 which provides strongly increased friction with the rolling membrane 1.

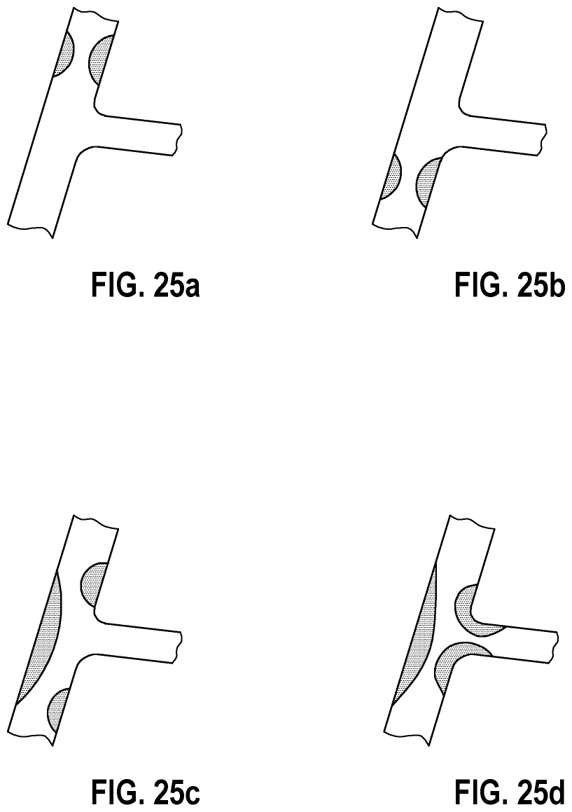

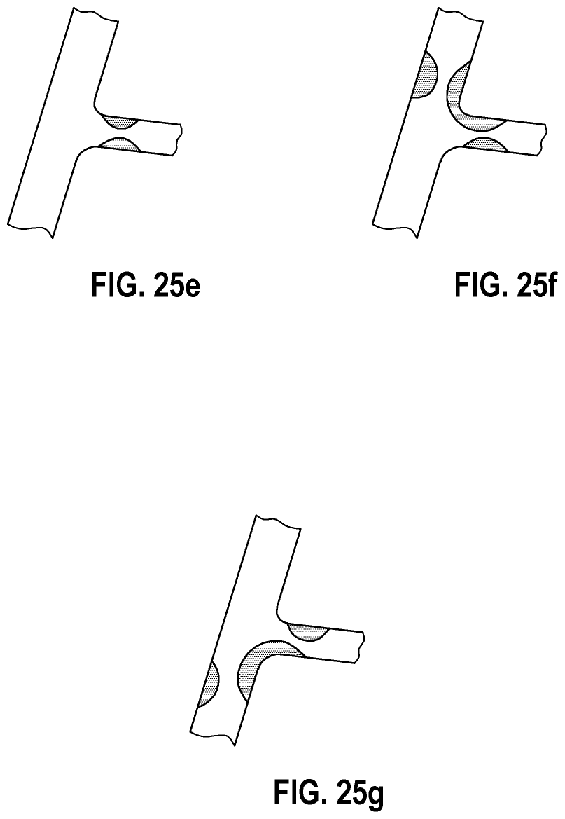

FIG. 25 shows an overview of potential locations of lesions which may need to accessible by a catheter according to the present invention. Typically, the rolling membrane 1 has sufficient stiffness to propagate “straight forward”. However, deviations from a straightforward propagation direction may require additional steering concepts as explained herein. Notably, a lesion may be located at a prebranch spot (FIG. 25a), a postbranch spot (FIG. 25b), in the parent vessel only (FIG. 25c), at a bifurcation (FIG. 25d), ostial (FIG. 25e), at a prebranch location and ostial (FIG. 25f) or at a postbranch location and ostial (FIG. 25g). It is to be understood that the dashed and/or dotted lines regarding the reference signs 4, 5, 6, 8, 9, 10, 16, 19, 20 can be seen as solid lines in terms of technical drawings and are only dashed and/or dotted for a better differentiation of the lines.

Throughout the Figures D denotes a distal direction and P a proximal direction. The distal direction is to be understood as the direction being further apart from a physician manipulating the catheter, with respect to the longitudinal extension of the catheter. The proximal direction is to be understood as the direction being closer to the physician manipulating the catheter, with respect to the longitudinal extension of the catheter. The distal direction may be opposite to the proximal direction.

Further examples of the present disclosure are provided below:

A catheter, including a rolling membrane; wherein the rolling membrane is adapted to roll in a longitudinal direction along a vessel of a patient (e.g. by at least 1 mm or by 1 cm to 20 cm).

A catheter, including a rolling membrane defining an inner volume that can be pressurized; and a guiding element arranged within the inner volume (for guiding the rolling membrane along a vessel of a patient).

The guiding element can be adapted to be slidable with respect to an inner side of the rolling membrane, such that the guiding element can remain at a distal portion of the rolling membrane as the rolling membrane is rolled out.

The guiding element can include a curved wire.

A catheter, including a rolling membrane; a retraction element without an inner lumen for retracting the rolling membrane from a rolled out state of the rolling membrane.

An end portion of the rolling membrane is preferably adapted to form a distal tip of the catheter when the rolling membrane is rolled out.

The retraction element can be attached to the end portion of the rolling membrane.

The guiding element can be adapted to steer the rolling membrane along the vessel as it is rolled out.

An outer diameter of the catheter is preferably smaller than a diameter of the vessel.

The rolling membrane can be adapted to form a helix-like shape along a longitudinal direction of the vessel.

A catheter, including a rolling membrane defining an inner volume that can be pressurized, wherein the rolling membrane is adapted such that, by pressurizing the inner volume, a stenosis in a blood vessel of a patient can be dilated via dilation by the rolling membrane.

An outer diameter of the catheter can be adapted to essentially fill the vessel of the patient in a radial direction.

The rolling membrane is preferably adapted such that a proximal end of the rolling membrane can be located outside of a body of a patient, as the rolling membrane is rolled out inside the body of the patient.

The rolling membrane is preferably at least partially provided with a non-uniform outer surface.

A radius of the rolling membrane, in a rolled out state, preferably varies along a longitudinal direction of the rolling membrane.

A catheter, including a rolling membrane adapted to be rolled out such as to form an elongated main portion and a distal end portion, wherein the elongated main portion is adapted to have a non-uniform cross-section.

A catheter, including a rolling membrane, wherein at least a portion of the rolling membrane can be folded inwards forming an inner side wall, wherein the inner side wall includes a drug layer, such that the drug layer is transported distally by rolling out the rolling membrane.

A catheter, including a rolling membrane, wherein the rolling membrane is at least partially provided with a non-uniform outer surface.

A catheter, including a rolling membrane adapted to be rolled out such as to form an elongated main portion having a cross-section that is smaller than a cross-section of a vessel, wherein a center line of the elongate main portion is adapted to twine around a center line of the vessel.

A catheter having a rolling membrane, wherein the catheter does not include an inner shaft.

A catheter, including a rolling membrane, wherein the rolling membrane is adapted to apply a force onto a functional element placed in a lumen at least in part formed by an inward-folded portion of the rolling membrane.

The rolling membrane is preferably adapted such that the force pushes the functional element in a distal direction of the catheter, when the rolling membrane is rolled out in the distal direction.

A method for dilating a stenosis in a vessel, the method including the steps of: inserting a catheter having a rolling membrane into the vessel; rolling out at least in part of the rolling membrane such as to place the rolling membrane in a proximity of the stenosis; and
pressurizing the rolling membrane such that the stenosis is dilated.

A method for forcing a guiding element into a stenosis region of a vessel, the method including: inserting a catheter having a rolling membrane into the vessel, wherein at least a portion of the guiding element is placed in a lumen at least in part formed by an inward-folded portion of the rolling membrane; rolling out at least a portion of the rolling membrane in a distal direction of the catheter, such that the guiding element is forced in the distal direction and into the stenosis region.

A catheter, including or consisting of a rolling membrane defining an inner volume that can be pressurized; a guiding element, optionally an outer shaft and optionally a retraction element, (wherein the rolling membrane is adapted to roll out in a longitudinal direction in a rolled out state, preferably along a vessel of a patient) wherein the guiding element is arranged within the inner volume for guiding the rolling membrane along a vessel of a patient.

The catheter includes the retraction element for retracting the rolling membrane (from a rolled out state of the rolling membrane, preferably into a non-rolled out state or a partially rolled out state of the rolling membrane).

The guiding element is preferably attached to the rolling membrane, preferably to an inner surface of the rolling membrane.

The guiding element and/or the retraction element can be attached to an (distal) end portion of the rolling membrane.

The guiding element and the retraction element can be attached to different locations of the (distal) end portion of the rolling membrane.

The catheter can have an outer diameter which is the same or smaller than the vessel to be inserted.

The rolling membrane can be made of an organic polymer, preferably a thermoplastic or a thermoplastic elastomer.

The rolling membrane can be made of a polyamide, a poly (dodecano-12-lactam), a polyether block amide or a thermoplastic polyurethane.

The guiding element and/or the retraction can lack an inner lumen.

The guiding element can be a guidewire, preferably a curved guidewire.

The guiding element and/or the retraction element is configured to not pierce or break through the rolling membrane.

The rolling membrane can be attached to the guiding element and/or the retraction element in a pressure-tight manner and/or in a fluid-tight manner.

The rolling membrane can be welded or glued to the guiding element and/or the retraction element.

The guiding element can be adapted to be slidable with respect to an inner side of the rolling membrane, such that the guiding element can remain at a distal portion of the rolling membrane as the rolling membrane is in the rolled out state.

The end portion of the membrane can be adapted to form a distal tip of the catheter when the rolling membrane is in the rolled out state.

The guiding element and/or the retraction element can be adapted to steer the rolling membrane along the vessel as the rolling membrane is in the rolled out state.

The rolling membrane can be adapted to form a helix-like shape along a longitudinal direction of the vessel.

The outer diameter of the catheter can be adapted to fill the vessel of the patient in a radial direction.

The rolling membrane can be adapted such that a proximal end of the rolling membrane can be located outside of a body of a patient, as the rolling membrane is in the rolled out state.

The rolling membrane can be adapted such that a distal end of the rolling membrane can be located inside the body of the patient, as the rolling membrane is in the rolled out state.

The rolling membrane can be at least partially provided with a non-uniform outer surface.

The rolling membrane can have spikes at its outer surface.

The rolling membrane can have (thermoformed) circumferential grooves, preferably helically arranged grooves, at its outer surface.

The rolling membrane can have (thermoformed) circumferential peaks, preferably helically arranged peaks, at its outer surface.

The outer diameter of the rolling membrane, in the rolled out state, can vary along the longitudinal direction of the rolling membrane.

The outer diameter of the rolling membrane, in the rolled out state, along the longitudinal direction of the rolling membrane can decrease from the proximal end of the rolling membrane to the distal end of the rolling membrane.

The outer diameter of the rolling membrane, in the rolled out state, along the longitudinal direction of the rolling membrane can continuously decrease from the proximal end of the rolling membrane to the distal end of the rolling membrane.

The outer diameter of the rolling membrane, in the rolled out state, along the longitudinal direction of the rolling membrane can decrease stepwise from the proximal end of the rolling membrane to the distal end of the rolling membrane.

The outer diameter of the rolling membrane, in the rolled out state, along the longitudinal direction of the rolling membrane can increase from the proximal end of the rolling membrane to the distal end of the rolling membrane.

The outer diameter of the rolling membrane, in the rolled out state, along the longitudinal direction of the rolling membrane can continuously increase from the proximal end of the rolling membrane to the distal end of the rolling membrane.

The outer diameter of the rolling membrane, in the rolled out state, along the longitudinal direction of the rolling membrane can increase stepwise from the proximal end of the rolling membrane to the distal end of the rolling membrane.

The outer diameter of the rolling membrane, in the rolled out state can be between 0.5 mm and 8.6 mm, or between 0.66 mm and 3.3 mm, or between 0.99 mm and 1.98 mm.

The rolling membrane can be a drug coated rolling membrane.

The rolling membrane can be a double-walled rolling membrane.

The rolling membrane when being pressurized can be extendable in longitudinal direction at a higher extent than in the radial direction.

The catheter can lack an inner shaft.

A catheter, including or consisting of a rolling membrane defining an inner volume that can be pressurized; a guiding element, an inner shaft, optionally an outer shaft and optionally a retraction element, (wherein the rolling membrane is adapted to roll out in a longitudinal direction into a rolled out state, preferably along a vessel of a patient) wherein the guiding element is arranged within the inner shaft and the inner shaft is attached to the rolling membrane.

The inner shaft can be reinforced by a metal, metal alloy or polymer braid.

The guiding element can be attached to the rolling membrane, preferably to an inner surface of the rolling membrane.

The guiding element and/or the retraction element can be attached to an (distal) end portion of the rolling membrane.

The guiding element and the retraction element can be attached to different locations of the end portion of the rolling membrane.

The catheter can have an outer diameter which is the same or smaller than the vessel to be inserted.

The rolling membrane can be made of an organic polymer, preferably a thermoplastic or a thermoplastic elastomer.

The rolling membrane can be made of a polyamide, a poly (dodecano-12-lactam), a polyether block amide or a thermoplastic polyurethane.

The guiding element and/or the retraction can lack an inner lumen.

The guiding element can be a guidewire, preferably a curved guidewire.

The guiding element and/or the retraction element can be configured to not pierce or break through the rolling membrane.

The rolling membrane can be attached to the guiding element and/or the retraction element in a pressure-tight manner and/or in a fluid-tight manner.

The rolling membrane can be welded or glued to the guiding element and/or the retraction element.

The guiding element can be adapted to be slidable with respect to an inner side of the rolling membrane, such that the guiding element can remain at a distal portion of the rolling membrane as the rolling membrane is in the rolled out state.

The end portion of the membrane can be adapted to form a distal tip of the catheter when the rolling membrane is in the rolled out state.

The guiding element and/or the retraction element can be adapted to steer the rolling membrane along the vessel as the rolling membrane is in the rolled out state.

The rolling membrane is adapted to form a helix-like shape along a longitudinal direction of the vessel (14).

The outer diameter of the catheter can be adapted to fill the vessel of the patient in a radial direction.

The rolling membrane can be adapted such that a proximal end of the rolling membrane can be located outside of a body of a patient, as the rolling membrane is in the rolled out state.

The rolling membrane can be adapted such that a distal end of the rolling membrane can be located inside the body of the patient, as the rolling membrane is in the rolled out state.

The rolling membrane can be at least partially provided with a non-uniform outer surface.

The rolling membrane can have spikes at its outer surface.

The rolling membrane can have (thermoformed) circumferential grooves, preferably helically arranged grooves, at its outer surface.

The rolling membrane can have (thermoformed) circumferential peaks, preferably helically arranged peaks, at its outer surface.

The outer diameter of the rolling membrane, in the rolled out state, can vary along the longitudinal direction of the rolling membrane.

The outer diameter of the rolling membrane, in the rolled out state, along the longitudinal direction of the rolling membrane can decrease from the proximal end of the rolling membrane to the distal end of the rolling membrane.

The outer diameter of the rolling membrane, in the rolled out state, along the longitudinal direction of the rolling membrane can continuously decrease from the proximal end of the rolling membrane to the distal end of the rolling membrane.

The outer diameter of the rolling membrane, in the rolled out state, along the longitudinal direction of the rolling membrane can decrease stepwise from the proximal end of the rolling membrane to the distal end of the rolling membrane.

The outer diameter of the rolling membrane, in the rolled out state, along the longitudinal direction of the rolling membrane can increase from the proximal end of the rolling membrane to the distal end of the rolling membrane.

The outer diameter of the rolling membrane, in the rolled out state, along the longitudinal direction of the rolling membrane can continuously increase from the proximal end of the rolling membrane to the distal end of the rolling membrane.

The outer diameter of the rolling membrane, in the rolled out state, along the longitudinal direction of the rolling membrane can increase stepwise from the proximal end of the rolling membrane to the distal end of the rolling membrane.

The outer diameter of the rolling membrane, in the rolled out state can be between 0.5 mm and 25 mm, or between 0.66 mm and 3.3 mm, or between 0.99 mm and 1.98 mm.

The rolling membrane can be a drug coated rolling membrane.

The rolling membrane can be a double-walled rolling membrane.

The rolling membrane when being pressurized can be extendable in longitudinal direction at a higher extent than in the radial direction.

The rolling membrane can be attached to the inner shaft.

The guiding element can have regions of different stiffness and/or roughness.

The guiding element can have an atraumatic distal tip.

The rolling membrane can be attached to at least a distal end region of the inner shaft.

The inner shaft can be (at least partially) located within the inner volume of the rolling membrane.

The rolling membrane can be pressurizable by a fluid which can be guided through the inner volume of the rolling membrane but not to the inner shaft.

The catheter can include the outer shaft.

The rolling membrane can be attached to the outer shaft.

The rolling membrane can be attached to at least a distal end region of the outer shaft.

The rolling membrane can be more flexible than the outer shaft.

The outer shaft can be reinforced by a metal, metal alloy or polymer braid.

The rolling membrane can have a smaller wall thickness than the outer shaft.

The rolling membrane can have a wall thickness of 10 μm to 90 μm, preferably 15 μm to 60 μm, 70-90.

The outer shaft can have a wall thickness of 100 μm to 500 μm.

The catheter can be a coronary catheter or a peripheral catheter.

The catheter can have a length of between 20 cm and 200 cm.

A catheter system can include the catheter according to any one of the examples and a flushing unit for flushing a fluid through the inner shaft, wherein the flushing unit is in fluid communication with the inner shaft.

A catheter system can include the catheter according to any one of the examples and a suction unit for sucking fluids, blood clots or debris from the surrounding of the catheter through the inner shaft, wherein the suction unit is in fluid communication with the inner shaft.

A catheter system can include the catheter according to any one of the examples and a drug delivery system for delivering at least one drug through the inner shaft, wherein the drug delivery system is in fluid communication with the inner shaft.

A catheter system can include the catheter according to any one of the examples and a stent delivery system for guiding a stent through the inner shaft.

A catheter system can include the catheter according to any one of the examples and a scoring element delivery system for guiding a scoring element through the inner shaft.

A catheter system can include the catheter according to any one of the examples and a microcatheter delivery system for guiding a microcatheter through the inner shaft.

A catheter system can include the catheter according to any one of the examples and a balloon catheter delivery system for guiding a balloon catheter through the inner shaft.

The catheter system can include the catheter according to any one of the examples and an embolic protection filter delivery system for guiding embolic protection filter through the inner shaft.

A method for operating the catheter according to any one of the examples pressurizes the inner volume of the rolling membrane with a fluid, preferably an biocompatible liquid, more preferably physiological saline.

The inner volume of the rolling membrane can be pressurized such that a stenosis in a blood vessel of a patient is dilated due to extension of the rolling membrane in the longitudinal direction.

A step of fabricating the rolling membrane can be by blown film extrusion, blow molding of extruded tubes, microextrusion or by attaching an elastomeric tube to an inner and outer shaft and pulling the elastomeric tube back into the outer shaft.

A method for treating a narrow stenosis with a (crossing) catheter with or without an inner and/or outer shaft includes a rolling membrane, wherein the catheter is inserted into the vessel and the rolling membrane is pressurized with a fluid, such that the rolling membrane rolls out in a longitudinal direction (D) into a rolled out state.

The fluid can be a biocompatible liquid, preferably physiological saline or a contrast medium.

The rolling membrane can be made of an organic polymer, preferably a thermoplastic or a thermoplastic elastomer.

The rolling membrane can be made of a polyamide, a poly (dodecano-12-lactam), a polyether block amide or a thermoplastic polyurethane.

The rolling membrane can be a double-walled rolling membrane.

The outer diameter of the rolling membrane, in the rolled out state can be between 0.5 mm and 2.7 mm, or between 0.66 mm and 2.0 mm, or between 0.99 mm and 1.98 mm.

The rolling membrane can have a wall thickness of 10 μm to 90 μm, preferably 15 μm to 60 μm, 70-90.

The catheter according can have an outer shaft and the outer shaft can have a wall thickness of 100 μm to 500 μm.

The inner volume of the rolling membrane can be pressurized with a fluid and a pressure of between 10 bar to 30 bar.

A catheter can include a rolling membrane defining an inner volume that can be pressurized; wherein the rolling membrane is adapted to roll out in a longitudinal direction (D) into a rolled out state, characterized in that the catheter does not include an outer shaft, an inner shaft and a guiding element.

A catheter cam include a rolling membrane defining an inner volume that can be pressurized; wherein the rolling membrane is adapted to roll out in a longitudinal direction (D) into a rolled out state, wherein the catheter further includes an inner shaft and/or an outer shaft, wherein the inner shaft and/or the outer shaft are reinforced by a metal braid or by reinforcing fibers.

A catheter, preferably for crossing a stenosis or a chronic total occlusion, can include a rolling membrane defining an inner volume that can be pressurized; wherein the rolling membrane is adapted to roll out in a longitudinal direction (D) into a rolled out state, wherein the catheter further includes an inner shaft and/or an outer shaft and optionally a guiding element, wherein the catheter has a catheter length of 80 cm to 200 cm, the rolling membrane has an outer diameter of between 1.5 mm to 5.0 mm and the rolling membrane has a length of between 4 cm to 60 cm, preferably of between 4 cm to 20 cm.

A catheter for medical applications (except for intubation) with or without an inner shaft and/or outer shaft can include a rolling membrane defining an inner volume that can be pressurized and optionally a guiding element, wherein the rolling membrane is adapted to roll out in a longitudinal direction (D) into a rolled out state, wherein the catheter has a catheter length of up to 200 cm, the rolling membrane has an outer diameter of between 0.5 mm to 1.20 cm, preferably 0.5 mm to 1.00 cm, more preferably between 1.0 mm to 5.0 mm, and the rolling membrane has a length of up to 100 cm, preferably of between 4 cm to 60 cm.

A catheter can include a rolling membrane defining an inner volume that can be pressurized; wherein the rolling membrane is adapted to roll out in a longitudinal direction (D) into a rolled out state, characterized in that the catheter further includes an inner shaft, and optionally an outer shaft, wherein the inner shaft is attached to the rolling membrane and wherein a medical device and/or a medical device delivery system and/or a drug and/or a drug delivery system and/or a contrast agent and/or an aspiration system is arranged/located within the inner shaft or can be guided through the inner shaft.

The catheter can lack a stent, a stent structure, a guiding element or a guidewire.

The catheter can include a medical device or be part of medical device delivery system that includes a sensor, an implantable cardioverter-defibrillator electrode, an implantable cardioverter-defibrillator lead, a nerve simulation device, an intravascular ultrasound (IVUS) system, an intervascular coil, a scoring element, a scoring balloon, an intravascular lithotripsy device, an aspiration device, an aneurysm embolization device, a needle, a camera, a catheter, preferably a microcatheter, an ablation catheter, a guiding catheter or a balloon catheter.

The catheter can include the outer shaft.

The catheter can lack an inner shaft.

The catheter can lack an outer shaft.

The rolling membrane can define an inner volume that can be pressurized and the rolling membrane can be adapted to roll out in a longitudinal direction (D) into a rolled out state.

The catheter can lack an outer shaft.

The catheter can include an inner shaft.

The rolling membrane can define an inner volume hat can be pressurized and the rolling membrane can be adapted to roll out in a longitudinal direction (D) into a rolled out state.

The rolling membrane can be attached to the outer shaft.

The rolling membrane can be attached to at least a distal end region of the outer shaft.

The rolling membrane can be more flexible than the outer shaft.

The rolling membrane can have a smaller wall thickness than the outer shaft.

The outer shaft can be reinforced, preferably by a metal braid, a metal alloy braid, a polymer braid or by reinforcing fibers.

The rolling membrane can be attached to the inner shaft, preferably to at least a distal end region of the inner shaft.

The rolling membrane can have a smaller wall thickness than the inner shaft.

The rolling membrane can be more flexible than the inner shaft.

The inner shaft can be reinforced, preferably by a metal braid, a metal alloy braid, a polymer braid or by reinforcing fibers.

The catheter can include a handle.

The handle can include a stopper or locking mechanism which is configured to avoid advancement of the rolling membrane in the longitudinal direction and/or rotation of the rolling membrane.

The handle can include a marker or a ruler.

The catheter can include the guiding element.

The guiding element can be arranged within the inner volume of the rolling membrane.

The guiding element can be a guidewire, preferably a curved guidewire.

The guiding element can be arranged within the inner shaft.

The guiding element can have regions of different stiffness and/or roughness.

The guiding element can have an atraumatic distal tip.

An outer diameter of the catheter can be the same or smaller than an inner diameter of a vessel to be inserted.

The rolling membrane can be at least partially provided with a non-uniform outer surface.

The rolling membrane can have spikes at its outer surface.

The rolling membrane can have circumferential grooves, preferably helically arranged grooves, at its outer surface.

The rolling membrane can have circumferential peaks, preferably helically arranged peaks, at its outer surface.

The outer diameter of the rolling membrane, in the rolled out state, along the longitudinal direction of the rolling membrane can decrease continuously or stepwise from the proximal end of the rolling membrane to the distal end of the rolling membrane.

The outer diameter of the rolling membrane, in the rolled out state, along the longitudinal direction of the rolling membrane can increase continuously or stepwise from the proximal end of the rolling membrane to the distal end of the rolling membrane.

The rolling membrane can include a drug or is at least partially covered by a drug coating layer.

The rolling membrane can be made of an organic polymer, preferably a thermoplastic or a thermoplastic elastomer.

The rolling membrane can be made of a polyamide, a poly (dodecano-12-lactam), a polyether block amide or a thermoplastic polyurethane.

The rolling membrane when being pressurized can be extendable in longitudinal direction at a higher extent than in the radial direction.

The inner shaft can be at least partially located within the inner volume of the rolling membrane.

The rolling membrane can be a double-walled rolling membrane.

A catheter with or without an inner shaft and/or an outer shaft can include a rolling membrane, and optionally a guiding element, for use in drug delivery, in crossing chronic total occlusions, in crossing (narrow) stenosis, in gastrointestinal interventions, in interventional oncology, in intravascular lithotripsy, in treating calcifications, in sheath assisted PVI procedures, in thrombectomy, in treating acute myocardial infarction, in renal denervation, in treating vein occlusions, in embolic protection device delivery, in reentry catheter procedures, in implantable cardioverter-defibrillator electrode delivery, in vascular interventions, preferably coronary vascular interventions or peripheral vascular interventions or in urology, preferably in urethra stricture treatment, wherein the rolling membrane defines an inner volume that can be pressurized and wherein the rolling membrane is adapted to roll out in a longitudinal direction (D) into a rolled out state.

Use of a catheter with or without an inner shaft and/or an outer shaft can include a rolling membrane, and optionally a guiding element, for use in drug delivery, in crossing chronic total occlusions, in crossing (narrow) stenosis, in gastrointestinal interventions, in interventional oncology, in intravascular lithotripsy, in treating calcifications, in sheath assisted PVI procedures, in thrombectomy, in treating acute myocardial infarction, in renal denervation, in treating vein occlusions, in embolic protection device delivery, in reentry catheter procedures, in implantable cardioverter-defibrillator electrode delivery, in vascular interventions, preferably coronary vascular interventions or peripheral vascular interventions or in urology, preferably in urethra stricture treatment, wherein the rolling membrane defines an inner volume that can be pressurized and wherein the rolling membrane is adapted to roll out in a longitudinal direction (D) into a rolled out state.

A catheter with or without an inner shaft and/or an outer shaft can include a rolling membrane, and optionally a guiding element, for use as access catheter, as support catheter, as guiding catheter, as guide extension catheter, as re-entry catheter, as introducer sheath or as scoring balloon catheter, wherein the rolling membrane defines an inner volume that can be pressurized and wherein the rolling membrane is adapted to roll out in a longitudinal direction (D) into a rolled out state.

Use of a catheter with or without an inner shaft and/or an outer shaft can include a rolling membrane, and optionally a guiding element, for use as access catheter, as support catheter, as guiding catheter, as guide extension catheter, as re-entry catheter, as introducer sheath or as scoring balloon catheter, wherein the rolling membrane defines an inner volume that can be pressurized and wherein the rolling membrane is adapted to roll out in a longitudinal direction (D) into a rolled out state.

A catheter with or without an inner shaft and/or an outer shaft can include a rolling membrane, and optionally a guiding element, for use as all-in-one device enabling approaching a lesion, preparing a lesion, pre-dilating the lesion, dilating the lesion, crossing the lesion, treating the lesion, wherein the rolling membrane defines an inner volume can be pressurized and wherein the rolling membrane is adapted to roll out in a longitudinal direction (D) into a rolled out state.

Use of a catheter with or without an inner shaft and/or an outer shaft can include a rolling membrane, and optionally a guiding element, as all-in-one device enabling approaching a lesion, preparing a lesion, pre-dilating the lesion, dilating the lesion, crossing the lesion, treating the lesion, wherein the rolling membrane defines an inner volume that can be pressurized and wherein the rolling membrane is adapted to roll out in a longitudinal direction (D) into a rolled out state.

A kit which can include the catheter according to any one of the preceding examples and directions for use.

A method for treating a stenosis using catheter according to any one of the examples can include pressurizing the inner volume of the rolling membrane with a fluid, such that the rolling membrane rolls out in a longitudinal direction (D) into a rolled out state and the stenosis in a blood vessel of a patient is dilated.

A method for treating a narrow stenosis or a chronic total occlusion with a catheter with or without an inner and/or outer shaft includes use of a rolling membrane, wherein the catheter is inserted into the vessel and the rolling membrane is pressurized with a fluid, such that the rolling membrane rolls out in a longitudinal direction (D) into a rolled out state and the stenosis in a blood vessel of a patient is dilated.

A method for re-entering a true arterial lumen by gaining a passage from a subintimal space along a chronic total occlusion can use a catheter according to one of the examples.

A method according to any one of the examples uses a fluid that is a biocompatible liquid, preferably physiological saline (which is an aqueous solution containing 0.9 wt % NaCl) or a contrast agent.

The rolling membrane can be transparent for electromagnetic radiation of between 300 nm to 1000 nm, preferably, 380 nm to 780 nm.

The rolling membrane can lack side branch(es).

A stenosis within the context of this application is a narrowing or constriction of a hollow organ or body lumen, for example of a blood vessel, a trachea, a ureter or a urethra or a biliary tract. Sometimes such a narrowing is called a stricture, for example in case of an urethral stricture. In case of blood vessels a stenosis may be due to arteriosclerosis, wherein the artery develops lesions, which lead to a narrowing of the artery due to the accumulation of atheromatous plaque. Atheromatous plaque mostly includes macrophage cells or debris, lipids, calcium and fibrous connective tissue. A stenosis has a length and a degree of severity (hardness) as well as level of narrowing down by percentage of the original body lumen or hollow organ diameter or volume. A narrow stenosis is a stenosis having a narrowing of 50% or more but less than 100% of the original body lumen or hollow organ diameter or volume. In case of a blood vessel a stenosis may be considered when 50% or more of the blood vessel wall cross-sectional area consists of atheromatous plaque.

A chronic total occlusion (CTO) is the complete obstruction of a coronary artery (narrowing of 100% of the coronary artery diameter or volume). CTO having soft CTO caps in the beginning can start aging and can get hard, fibrous CTO caps with time.

An access or crossing catheter is a catheter used for crossing a lesion or a stenosis. An access catheter does not have the ability to cross a CTO alone due to a lack of pushability.

A crossing total occlusion catheter is a catheter used for crossing a CTO, preferably by recanalization of a CTO via a true lumen. A crossing total occlusion catheter has a higher pushability than the access catheter. Crossing total occlusion catheters can be categorized as either intraluminal, subintimal or re-entry devices. A re-entry crossing total occlusion catheter enters a true lumen from a subintimal space when combined with/without a guidewire in a chronic total occlusion.

A support catheter is a catheter (introducer) sheath. A support catheter may be 6 cm to 8 cm long and may have a diameter of 6 Fr to 9 Fr (1 Fr (French) is defined as being ⅓ mm). A support catheter may be a single lumen catheter (typically placed in a central vein). A combination of a rolling membrane catheter and a puncture needle is defined as introducer sheath. A support catheter is a catheter which is used during peripheral vascular intervention (PVI) procedures for guidewire support to enable lesion crossing, as well as for guidewire exchanges.

A guiding catheter is a catheter which is used during coronary interventions to provide access to the coronary ostium and support equipment deliver. In percutaneous coronary interventions (CVI) the guiding catheters selection is based on the vessel diameter, the tortuosity, the degree of calcification, the size of the ascending aorta, ostial orientation and the planned complexity of intervention.

A guide extension catheter is a catheter that is used to extend the length of the guiding catheter. A guide extension catheter may have a diameter which is 1 Fr smaller than the diameter of the guiding catheter.

Intravascular Lithotripsy is a device that modifies calcified lesions via calcium fracture in coronary and peripheral vessels.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A catheter configured to cross a stenosis or a chronic total occlusion, comprising:
a rolling membrane defining an inner volume that can be pressurized, the rolling membrane being configured to roll out in a longitudinal direction (D) into a rolled out state, and
an inner shaft and/or an outer shaft, wherein the catheter comprises a catheter length of 80 cm to 200 cm, the rolling membrane comprises an outer diameter of between 1.5 mm to 5.0 mm and the rolling membrane comprises a length of between 4 cm to 60 cm.

2. (canceled)

3. A catheter comprising:
a rolling membrane defining an inner volume that can be pressurized, the rolling membrane being configured to roll out in a longitudinal direction (D) into a rolled-out state:
an inner shaft attached to the rolling membrane; and
a medical device and/or medical device delivery system and/or a drug and/or a drug delivery system and/or a contrast agent and/or an aspiration system arranged within the inner shaft.

4. The catheter according to claim 3, comprising no stent, no stent structure, no guiding element and no guidewire.

5. The catheter according to claim 3, wherein the medical device comprises a sensor, an implantable cardioverter-defibrillator electrode, an implantable cardioverter-defibrillator lead, a nerve simulation device, an intervascular coil, a scoring element, a scoring balloon, an intravascular lithotripsy device, an aspiration device, an aneurysm embolization device, a needle, an intravascular ultrasound (IVUS) system, a camera, a catheter, a microcatheter, an ablation catheter, a guiding catheter or a balloon catheter.

6. The catheter according to claim 1, comprising an outer shaft.

7-14. (canceled)

15. The catheter according to claim 1, wherein the rolling membrane is more flexible than the outer shaft or the inner shaft.

16. The catheter according to claim 1, wherein the rolling membrane has a smaller wall thickness than the outer shaft or the inner shaft.

17. The catheter according to claim 1, wherein the outer shaft or the inner shaft is reinforced by a metal braid, a metal alloy braid, a polymer braid or by reinforcing fibers.

18-21. (canceled)

22. The catheter according to claim 1, comprising a handle.

23. The catheter according to claim 22, wherein the handle comprises a stopper or locking mechanism configured to avoid advancement of the rolling membrane in the longitudinal direction and/or rotation of the rolling membrane.

24. The catheter according to claim 22, wherein the handle comprises a marker or a ruler.

25. The catheter according to claim 1, wherein the catheter comprises a guiding element.

26. (canceled)

27. The catheter according to claim 25, wherein the guiding element comprises a guidewire.

28. (canceled)

29. The catheter according to claim 25, wherein the guiding element comprises regions of different stiffness and/or roughness.

30. The catheter according to claim 25, wherein the guiding element comprises an atraumatic distal tip.

31-32 (canceled)

33. The catheter according to claim 1, wherein the rolling membrane comprises spikes, circumferential grooves, and/or circumferential peaks at its outer surface.

34-35. (canceled)

36. The catheter according to claim 1, wherein an outer diameter of the rolling membrane, in the rolled out state, along the longitudinal direction decreases continuously or stepwise from a proximal end of the rolling membrane to a distal end of the rolling membrane.

37. (canceled)

38. The catheter according to claim 1, wherein the rolling membrane comprises a drug or is at least partially covered by a drug coating layer.

39-40. (canceled)

41. The catheter according to claim 1, wherein the rolling membrane is configured to be extendable in the longitudinal direction at a higher extent than in a radial direction.

42. The catheter according to claim 1, wherein the inner shaft is at least partially located within the inner volume of the rolling membrane.

43. (canceled)

44. A method for using a catheter according to claim 1, comprising use in drug delivery, in crossing chronic total occlusions, in crossing stenosis, in gastrointestinal interventions, in interventional oncology, in intravascular lithotripsy, in treating calcifications, in sheath assisted PVI procedures, in thrombectomy, in treating acute myocardial infarction, in aneurysm treatment, in renal denervation, in treating vein occlusions, in embolic protection device delivery, in reentry catheter procedures, in implantable cardioverter-defibrillator electrode delivery, in vascular interventions, preferably coronary vascular interventions or peripheral vascular interventions or in urology, preferably in urethra stricture treatment, wherein the rolling membrane defines an inner volume that can be pressurized and wherein the rolling membrane is configured to roll out in the longitudinal direction (D) into the rolled out state.

45-52. (canceled).
